SEQUENCE LISTING (37 sequences)

SEQ ID NO: 1 - DNA, 21 bp, Artificial Sequence
  Description of Artificial Sequence Synthetic oligonucleotide
aatgtgctatctggatcctag

SEQ ID NO: 2 - DNA, 21 bp, Artificial Sequence
  Description of Artificial Sequence Synthetic oligonucleotide
ctgctcgaaatcttacgcaaa

SEQ ID NO: 3 - DNA, 21 bp, Artificial Sequence
  Description of Artificial Sequence Synthetic oligonucleotide
aaggacggtgttcttggtcaa

SEQ ID NO: 4 - DNA, 21 bp, Artificial Sequence
  Description of Artificial Sequence Synthetic oligonucleotide
ctcgcataggcattcccagta

SEQ ID NO: 5 - DNA, 21 bp, Artificial Sequence
  Description of Artificial Sequence Synthetic oligonucleotide
atcgatcctatgttcaatcta

SEQ ID NO: 6 - DNA, 21 bp, Artificial Sequence
  Description of Artificial Sequence Synthetic oligonucleotide
cagatccgtgtggatcatgca

SEQ ID NO: 7 - DNA, 21 bp, Artificial Sequence
  Description of Artificial Sequence Synthetic oligonucleotide
tgggacgtttgtagaacctga

SEQ ID NO: 8 - DNA, 21 bp, Artificial Sequence
  Description of Artificial Sequence Synthetic oligonucleotide
accgacgagcaggcactggaa

SEQ ID NO: 9 - DNA, 21 bp, Artificial Sequence
  Description of Artificial Sequence Synthetic oligonucleotide
caaggaataatttctgatcca

SEQ ID NO: 10 - DNA, 20 bp, Artificial Sequence
  Description of Artificial Sequence Synthetic primer
tgatagccaaattgagacaa

SEQ ID NO: 11 - DNA, 20 bp, Artificial Sequence
  Description of Artificial Sequence Synthetic primer
ttcagggtgaggaagtaaaa

SEQ ID NO: 12 - DNA, 20 bp, Artificial Sequence
  Description of Artificial Sequence Synthetic primer
gaaggaaaaaatccagctga

SEQ ID NO: 13 - DNA, 23 bp, Artificial Sequence
  Description of Artificial Sequence Synthetic primer
gaataaaatttgtctttcctgcc

SEQ ID NO: 14 - DNA, 21 bp, Artificial Sequence
  Description of Artificial Sequence Synthetic primer
aggatttgagcggaagaacga

SEQ ID NO: 15 - DNA, 22 bp, Artificial Sequence
  Description of Artificial Sequence Synthetic primer
acaccagcaaaatgtactcagc

SEQ ID NO: 16 - DNA, 21 bp, Artificial Sequence
  Description of Artificial Sequence Synthetic primer
ctgtgggttagggatggactg

SEQ ID NO: 17 - DNA, 21 bp, Artificial Sequence
  Description of Artificial Sequence Synthetic primer
gtaaagtgggtggacgttgca

SEQ ID NO: 18 - DNA, 21 bp, Artificial Sequence
  Description of Artificial Sequence Synthetic primer
gacaccaaacccaatcaccac

SEQ ID NO: 19 - DNA, 21 bp, Artificial Sequence
  Description of Artificial Sequence Synthetic primer
gttcaacctgccactgacctt

SEQ ID NO: 20 - DNA, 24 bp, Artificial Sequence
  Description of Artificial Sequence Synthetic primer
ccttctgtgttggcattgtctatc

SEQ ID NO: 21 - DNA, 22 bp, Artificial Sequence
  Description of Artificial Sequence Synthetic primer
caagctcttgccagaaggtcag

SEQ ID NO: 22 - DNA, 23 bp, Artificial Sequence
  Description of Artificial Sequence Synthetic primer
ggagatttggcaggcataccttg

SEQ ID NO: 23 - DNA, 22 bp, Artificial Sequence
  Description of Artificial Sequence Synthetic primer
ctcttccacctcctgcttcaga

SEQ ID NO: 24 - DNA, 20 bp, Artificial Sequence
  Description of Artificial Sequence Synthetic primer
tagccaaccgagagacaagc

SEQ ID NO: 25 - DNA, 23 bp, Artificial Sequence
  Description of Artificial Sequence Synthetic primer
tgtgctcccaccactaataaaag

SEQ ID NO: 26 - DNA, 21 bp, Artificial Sequence
  Description of Artificial Sequence Synthetic primer
tgtaaaccctgcagatcctga

SEQ ID NO: 27 - DNA, 21 bp, Artificial Sequence
  Description of Artificial Sequence Synthetic primer
tggaggatgctgaaatccaat

SEQ ID NO: 28 - DNA, 21 bp, Artificial Sequence
  Description of Artificial Sequence Synthetic primer
aactttcgatggtagtcgccg

SEQ ID NO: 29 - DNA, 21 bp, Artificial Sequence
  Description of Artificial Sequence Synthetic primer
ccttggatgtggtagccgttt

SEQ ID NO: 30 - DNA, 27 bp, Artificial Sequence
  Description of Artificial Sequence Synthetic primer
aaagaattcatgtcctctgaagaagga

SEQ ID NO: 31 - DNA, 27 bp, Artificial Sequence
  Description of Artificial Sequence Synthetic primer
aaactcgagtcagttgtcataattgtc

SEQ ID NO: 32 - DNA, 20 bp, Artificial Sequence
  Description of Artificial Sequence Synthetic oligonucleotide
gcgtattatagccgattaac

SEQ ID NO: 33 - DNA, 20 bp, Artificial Sequence
  Description of Artificial Sequence Synthetic oligonucleotide
gtttctaaaaatacaccagc

SEQ ID NO: 34 - DNA, 54 bp, Artificial Sequence
  Description of Artificial Sequence Synthetic oligonucleotide
ccattaaagagtgttcgcagacaaagtttctaaaaatacaccagcaaaatgtac

SEQ ID NO: 35 - DNA, 160 bp, Artificial Sequence
  Description of Artificial Sequence Synthetic polynucleotide
  modified_base (3)..(3): a, c, t, or g
acnttattgcgtcgtatttgtgattgagctgagtacattttgctggtgtatttttagaaactttgtctgcgaaaggctcgttgctttatttctccatttcccgttcctggggagttgggaggcagaatttccggttccatttctggagagtggcacgggg

SEQ ID NO: 36 - DNA, 27 bp, Artificial Sequence
  Description of Artificial Sequence Synthetic oligonucleotide
tgtatttttagaaactttgtctgcgaa

SEQ ID NO: 37 - DNA, 63 bp, Artificial Sequence
  Description of Artificial Sequence Synthetic oligonucleotide
gtcgtatttgtgattgaagctgagtacattttgctggtgtatttttagaaactttgtctgcga

(19) United States
(12) Patent Application Publication
(10) Pub. No.: US 2023/0374508 A1
(43) Pub. Date: Nov. 23, 2023

(54) Compositions and Methods Targeting circ2082 for the Treatment of Cancer

(71) Applicant: The Brigham and Women`s Hospital, Inc., Boston, MA (US)

(72) Inventors: Jakub Godlewski, Boston, MA (US); Agnieszka Bronisz, Boston, MA (US); Ennio Antonio Chiocca, Boston, MA (US)

(21) Appl. No.: 18/031,054

(22) PCT Filed: Oct. 13, 2021
(86) PCT No.: PCT/US2021/054763

§ 371 Date: Apr. 10, 2023

Related U.S. Application Data
(60) Provisional application No. 63/091,251, filed on Oct. 13, 2020.

(51) Int. Cl. C12N 15/113; A61P 35/00

(52) U.S. Cl. CPC C12N 15/113; A61P 35/00; C12N 2310/11; C12N 2310/341; C12N 2310/30

(57) ABSTRACT

Described herein are compositions comprising inhibitory nucleic acids targeting a circularization junction site of circ2082, and methods of using those compositions for treating cancers, e.g., brain cancer such as glioblastoma.

### CLAIM OF PRIORITY

This application claims the benefit of U.S. Provisional Application Ser. No. 63/091,251, filed on Oct. 13, 2020. The entire contents of the foregoing are incorporated herein by reference.

### FEDERALLY SPONSORED RESEARCH OR DEVELOPMENT

This invention was made with Government support under Grant No. CA176203 awarded by the National Institutes of Health. The Government has certain rights in the invention.

### TECHNICAL FIELD

Described herein are compositions comprising inhibitory nucleic acids targeting a circularization junction site of circ2082, and methods of using those compositions for treating cancers, e.g., brain cancer such as glioblastoma.

### BACKGROUND

The assortment of cellular microRNAs (“microRNAome”) is a vital readout of cellular homeostasis, but the mechanisms that regulate the microRNAome are poorly understood.

### SUMMARY

Provided herein are inhibitory nucleic acids, e.g., comprising 10-20, 10-30, 10-40, 20-30, 20-40, 30-40, 30-50, or 10-50 nucleotides in length, comprising a sequence complementary to at least 10 consecutive nucleotides (nts) of SEQ ID NO:37, preferably comprising a sequence complementary to at least nts 48-49 or 47-50 of SEQ ID NO:37, plus additional nts on one or both ends.

In some embodiments, the inhibitory nucleic acid comprises at least 10, 11, 12, 13, 14, 15, 16, 17, 18, 19, or all 20 consecutive nucleotides of GTTTCTAAAAATACACCAGC (SEQ ID NO:33).

In some embodiments, the inhibitory nucleic acid is or comprises an antisense RNA oligonucleotide; antisense DNA oligonucleotide; chimeric antisense oligonucleotide; short, hairpin RNA (shRNA); or single- or double-stranded short interfering RNA (siRNA) for RNA interference (RNAi).

In some embodiments, the inhibitory nucleic acid comprises one or more modifications. In some embodiments, the one or more modifications comprise one or more modified bonds or bases, and/or conjugation to one or more moieties or conjugates that enhance the activity, cellular distribution, or cellular uptake of the oligonucleotide.

In some embodiments, the modified bonds comprise amide backbone; morpholino backbone; or peptide nucleic acid (PNA) backbone; wherein the modified bases comprise locked nucleic acids, phosphorothioate, methylphosphonate, peptide nucleic acids; and/or the conjugated moiety is a cholesterol, α-tocopherol, polyethulene glycol (PEG), biotin, or nanoparticle. In some embodiments, the chimeric antisense oligonucleotide is a gapmer or mixmer or a DNA/RNA heteroduplex oligonucleotide (HDO).

In some embodiments, the inhibitory nucleic acid comprises SEQ ID NO:33.

Also provided herein are compositions, e.g., pharmaceutical compositions, comprising the inhibitory nucleic acids described herein, and a pharmaceutically effective carrier.

Additionally, provided herein are methods for treating a cancer in a subject. The methods comprise administering to the subject a therapeutically effective amount of an inhibitory nucleic acid, preferably comprising 10-50 nucleotides in length, comprising a sequence complementary to at least 10 consecutive nucleotides (nts) of SEQ ID NO:37, preferably comprising a sequence complementary to at least nts 48-49 or 47-50 of SEQ ID NO:37, plus additional nts on one or both ends. Also provided are the nucleic acids and compositions described herein for use in treating cancer in a subject.

In some embodiments, the inhibitory nucleic acid comprises at least 10, 11, 12, 13, 14, 15, 16, 17, 18, 19, or all 20 consecutive nucleotides of GTTTCTAAAAATACACCAGC (SEQ ID NO:33).

In some embodiments, the inhibitory nucleic acid is or comprises an antisense RNA oligonucleotide; antisense DNA oligonucleotide; chimeric antisense oligonucleotide; short, hairpin RNA (shRNA); or single- or double-stranded short interfering RNA (siRNA) for RNA interference (RNAi).

In some embodiments, the inhibitory nucleic acid comprises one or more modifications. In some embodiments, the one or more modifications comprise one or more modified bonds or bases, and/or conjugation to one or more moieties or conjugates that enhance the activity, cellular distribution, or cellular uptake of the oligonucleotide. In some embodiments, the modified bonds comprise amide backbone; morpholino backbone; or peptide nucleic acid (PNA) backbone; wherein the modified bases comprise locked nucleic acids, phosphorothioate, methylphosphonate, peptide nucleic acids; and/or the conjugated moiety is a cholesterol, α-tocopherol, polyethulene glycol (PEG), or biotin. In some embodiments, the chimeric antisense oligonucleotide is a gapmer or mixmer or a DNA/RNA heteroduplex oligonucleotide (HDO). In some embodiments, the inhibitory nucleic acid comprises SEQ ID NO:33.

In some embodiments, the cancer is a solid tumor.

In some embodiments, the cancer is brain cancer, optionally glioblastoma brain breast; prostate; pancreatic; hepatic; lung; kidney; skin; head and neck; bladder; ovarian; or colon cancer.

Also provided herein are methods to prevent, treat or slow the progress of cancer or any tumorigenic condition in a patient comprised of administering to said patient a therapeutically effective amount of an agent that inhibits the suppression of the cellular microRNAome caused by aberrant localization of one, or all, of the component(s) of the microRNAome processing complex to the nucleus of a cancer cell.

In some embodiments, the component of the microRNAome processing complex is DICER. In some embodiments, said agent binds to various components of said DICER complex or interactome thereby shifting DICER subcellular localization from the nucleus to the cytoplasm in said cancer cell. In some embodiments, said components of the nuclear DICER complex or interactome include, but are not limited to, RNA binding proteins and circular RNA. In some embodiments, the RNA binding protein is RBM3.

In some embodiments, the circular RNA is circ2082.

In some embodiments, said agent includes, but is not limited to, a small molecule, an antisense oligonucleotide, an antibody, or an siRNA.

In some embodiments, the cancer or tumor is characterized by an abundance or enrichment of cancer stem-like cells (CSC). In some embodiments, the CSC enriched-tumor is in brain. In some embodiments, the brain tumor is glioblastoma.

Additionally, provided herein are antisense oligonucleotides comprised of a nucleotide sequence targeted to the sense strand of a nucleotide fragment encoding circ2082. In some embodiments, the sequence of the oligonucleotide comprises 20 consecutive nucleotides of GTTTCTAAAAATACACCAGC (SEQ ID NO:33). Also provided are pharmaceutical compositions comprised of a therapeutically effective amount of the antisense oligonucleotides and a pharmaceutically acceptable carrier.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-B. Mature, but not precursor, microRNAome is suppressed in glioblastomas and GSCs. Mature microRNAome but not microRNA precursors distinguished between glioblastoma patients and healthy individuals. Principal component analysis of mature (A) and precursors (B).

FIGS. 2A-E. DICER is retained in the nucleus of GSCs, forming a complex with RBM3 protein. A: Representative Western blotting of DICER in cytosolic (C) and nuclear (N) fractions from NPCs and GSCs. B. C: Representative Coomassie Blue staining (B) and mass-spectroscopy peaks of DICER-associated proteins (C) using GSC nuclear (top) and cytosolic (bottom) extract IP with either IgG (black peaks), or DICER antibodies (grey peaks) (n=3). D: Representative Western blots of nuclear lysate inputs and IP obtained by IgG and DICER antibodies. E: Representative Western blotting of cytosolic (C) and nuclear (N) fractions from NPCs and GSCs. Loading and fraction purity controls are shown in FIG. 2A.

FIGS. 3A-L. DICER/RBM3 forms complex with circ2082. A: RIP of nuclear lysates with DICER antibodies followed by RNA isolation, RNase treatment, and qPCR (n=3) using indicated primers. B: Agarose gel of PCR products using cDNA or gDNA and indicated primers. C: RIP of nuclear extracts from GSC using IgG and DICER antibodies, qPCR analysis with mean±SD. D, E, F: Coomassie Blue staining (D), mass-spectroscopy peaks of circ2082-associated proteins (E), and Western blot (F) using GSC protein extracts upon RAP with control or anti-circ2082 probes (n=3). G, H, I: RIP of lysates from GSC transfected with GFP or GFP-RBM3 vector. Protein inputs analyzed by Western blot (G), RNA profile by Agilent Bioanalyzer (H), and circ2082 enrichment in RIP by qPCR with mean±SD (I). J, K, L: qPCR analysis of circ2082 (top), and linear MALAT1 (bottom) with mean±SD in: NPCs vs. M GSCs (50.1-fold, and 2.5-fold, respectively), and NPCs vs. P GSCs (150.2-fold, and 3.8-fold, respectively), (n=4 per group) (J); adjacent brain vs. glioblastoma (2.3-fold, and 1.4-fold paired, respectively), matching lines identify pairs, (n=5) (K); and NPCs vs. GSCs (12.8-fold, and 3.9-fold, respectively), and NPC vs. other cancers (27.1-fold, and 4.6-fold, respectively), (non-malignant cells, n=6, GSCs, n=3 per subtype, other cancers, n=9) (L); p-values: * <0.05, ** <0.01, *** <0.001, **** <0.0001.

FIGS. 4A-D. The knockdown of circ2082 results in a widespread de-repression of GSC microRNAome. A: qPCR analysis for circ2082 or linear MALAT1 in GSCs with mean±SD. GSC (green, proneural, red, mesenchymal, n=3 per subtype) were transfected with ASO control or circ2082. B: A scatter plots for GSCs (M GSC: left; P GSC: right) transfected with ASO control or circ2082 (n=3 each) based on levels of 756 mature microRNAs. C: Scatter plots for GSCs (M GSC: left; P GSC: right) transfected with ASO control or circ2082 (n=3 each) based on levels of 22 precursor. D: Representative Western blotting of cytosolic (C) and nuclear (N) fractions of GSCs (n=4) transfected with ASO control or circ2082.

FIGS. 5A-D. The knockdown of circ2082 mitigates tumorigenicity of GSCs. A, B: Representative sphere frequency assays using linear regression plot. GSC (n=3 per subtype) were transfected with ASO control or circ2082. P-values are indicated. C, D: Kaplan-Meier curves of mice injected intracranially (n=5 per group) with GSCs (left: mesenchymal, right: proneural) transfected with ASO control or circ2082. P-values are indicated.

FIGS. 6A-F. The circ2082-dependent footprint is mediated via the re-arrangements of the microRNAome. A: Heatmap displaying the clustering of expression correlation of genes (n=1697) and selected microRNAs using the TCGA database. The correlation value (dark grey-negative, light grey-positive) and annotations of profile similarity (bottom bar) are shown. B: Kaplan-Meier curves survival analysis based on genes associated with 6 microRNA in the TCGA database and stratified according to their cluster membership (see 6A). C: Volcano plot of genes (n=1697, see panel a) for M GSCs (n=3, left), P GSCs (n=3, middle), and all GSCs (n=6, right) transfected with ASO control or circ2082 are shown. The selected de-regulated targets are shown. Lines indicate a p-value and fold difference cutoffs. D, E: qPCR analysis for indicated mRNAs upon the treatment by ASO circ2082 or tumor-suppressive microRNAs (D) and ASO circ2082 or oncogenic anti-microRNAs (E) in GSCs (n=3 per subtype). F: Kaplan-Meier curves survival analysis using TCGA database and stratified according to their cluster membership after circ2082 knock-down in M GSCs (left), P GSCs (middle), and all GSCs (right).

FIGS. 7A-F. Circ2082 that originates from MALAT1 binds directly to RBM3 and forms complex with DICER. A: The schematic representation of the experimental flowchart. Protein-RNA immunoprecipitation (RIP) of UV-crosslinked GSC nuclear lysates with DICER antibodies was followed by RNA isolation and analysis by Total RNA Platform Agilent Bioanalyzer. The representative RNA profile is shown (n=2). Arrows indicate circ2082 and small RNA peaks. The ladder indicates RNA size (nt) on the left. B: The schematic representation of linear MALAT1 and circularization event leading to the emergence of circ2082 (top). Primers used throughout the study to distinguish between linear MALAT1 and circ2082 are marked by arrows. The junction site sequence (SEQ ID NO:35) and sequencing result (SEQ ID NO:36) are shown (bottom); N is any of GATC. C-D: Western blot analysis of DICER (C) and RBM3 (D) in the whole-cell lysate of GSC (n=5) upon their respective siRNA-mediated knockdown using indicated antibodies is shown. Both antibodies were used as a loading control upon the knockdown of their respective protein partners; arrows indicate DICER and RBM3 bands. Corresponding molecular weight is shown on the left. E: Box scatter plot of qPCR analysis of circ2082 expression with mean±SD, and relative quantification scale is shown (n=5). GSCs were transfected with control, DICER, or RBM3 siRNA. F: Western blot analysis of DICER and RBM3 in GSCs transfected with control, DICER, and/or RBM3 siRNA. UV-crosslinked GSC nuclear lysate inputs and RNA Antisense Purification (RAP) with the anti-circ2082 probe are shown; arrows indicate DICER and RBM3 bands. Corresponding molecular weight is shown on the left.

FIGS. 8A-D. ASO treatment that silences circ2082 but not MALAT1 affects mature but not precursor microRNAs. A: Box plot of qPCR analysis for circ2082 or linear MALAT1 with mean±SD and relative quantification scale is shown. GSC (of indicated subtypes: red—M GSCs, green—P GSCs, n=3 per subtype) were transfected with control siRNA or MALAT1 siRNA. B: QPCR analysis of circ2082 and linear MALAT1 relative percentage of expression in the nuclear and cytoplasmic fraction of GSCs. C, D: Box plot of qPCR analysis for indicated mature microRNAs (d) and microRNA precursors (e) with mean±SD and relative quantification scale is shown. GSC (of indicated subtypes, n=3 per subtype) were transfected with ASO control or circ2082.

FIGS. 9A-C. ASO-mediated knockdown of circ2082 mitigates tumorigenicity of GSCs in vitro and in vivo. A: Representative micrographs of GSCs (n=3 per subtype) transfected with ASO control or circ2082 (scale bars: 500 μm (left)), and box scatter plot of mean±SD of sphere frequency, and volume (right) are shown (lines identify matching pairs, the p-value is indicated). B: Representative images of brains with M GSC-originated tumors are shown (left). Relative quantification of tumor volume 10 days post-implantation is shown (right). Data are shown as mean±SD (matching pairs identified by lines; p-value is indicated). C: Representative images of sections of M GSC-originated tumors are shown. GFP-positive M GSCs were transfected with Cy5-labeled ASO control or circ2082, scale bars: 2 mm (top), 250 μm (bottom).

FIGS. 10A-C. The expression of circ2082/microRNAome-controlled genes predict outcome of glioblastoma patients in subtype-independent fashion. A-C: Kaplan-Meier curves for survival analysis of (A) GLI2, JARID; (B) CCNG1, SART3; (C) MET and PDCD4 in TCGA dataset of glioblastoma patients. The p-value is indicated.

### DETAILED DESCRIPTION

MicroRNAs—short non-coding RNA molecules that control the expression/activity of a multitude of protein-coding genes, populate the human genome with high frequency. Despite the ever-growing number of putative microRNAs, the number of high-confidence, validated loci, has been recently approximated to be ˜2,300 (1). The number of putative protein-coding mRNAs that can be targeted via microRNA complementarity has been estimated at ˜18,000, a number that approximates that of the protein-coding transcriptome (2). Quantitatively, this would imply that most of the cell transcriptome, and thus, its proteome, is under microRNA surveillance.

The biogenesis and maturation of microRNAs have been thoroughly investigated since their discovery in the 1990s. MicroRNA genes embedded either within introns of protein-coding genes (intronic) or between them (intergenic) are transcribed first into long primary transcripts (pri-microRNAs) that are then processed in the nucleus into ˜80 nucleotide precursors (pre-microRNAs) forming hairpin-like structures. Pre-microRNAs are transported into the cytoplasm where an enzymatic complex consisting of Endoribonuclease Dicer (DICER, encoded by the gene DICER1) and its co-factors cleave them further into short ˜20 nucleotide mature microRNAs. These mature microRNAs can then regulate mRNA expression by loading onto the RISC protein complex that serves as an mRNA target seeker based on the complementarity between a 6-8 nucleotide-long sequence within the microRNA known as the “seed” and its target site located usually within the 3′UTR of mRNA (3). This interaction results in the cleavage of mRNA by RISC and/or less efficient translation at ribosomes, resulting in suppressed expression of the targeted gene. Although DICER-independent microRNAs exist (4), the vast majority of microRNAs rely on DICER for their processing, thus making the entire pathway vulnerable to malfunction if DICER activity is targeted.

The assortment of microRNAs expressed in the cell at any given time, the microRNAome, varies considerably between tissues, cell types, developmental and pathological stages, and upon response to stressors or stimuli. Importantly, microRNAs, due to their ability to fine-tune scores of genes, have been recognized as master guardians of cell fate decisions and terminal differentiation (5). As malignant transformation can be perceived as a faulty execution of cell-fate choices or as de-differentiation that went awry at cellular backstops, the discovery that the microRNAome of the cancer cell is very much different than that of its cell-of-origin counterpart (6) was, perhaps with the benefit of hindsight, not particularly surprising.

Cancer-specific signatures exist in most, if not nearly all, molecular readouts (transcriptomes, proteomes, metabolomes, etc.) of the cell. What makes the cancer microRNAome unique when compared to other classes of molecules (including other non-coding RNAs) is the persistent pattern of the de-regulation. In essence, most microRNAs are suppressed, while relatively few are over-expressed (6). One possible explanation is that most microRNAs impose programs of terminal differentiation, and tumor cells evade these programs by microRNAome suppression (6).

Possible mechanistic explanations for this observation of cancer-specific global suppression of the microRNAome are few. Chromosomal anomalies and altered epigenetic landscapes, both common in malignancies, would result in changes that would be more localized across the genome or would affect other players of the transcriptome similarly. Transcriptional de-regulation (again, frequent in transformed cells), on the other hand, functions mainly on a “case-by-case” fashion and thus cannot adequately explain this sweeping suppression of microRNAs. These considerations winnow the field of potential mechanistic culprits. Could disorders in the processing/maturation of microRNAs, primarily controlled by a single canonical pathway, be the wrongdoer in the observed cancer-specific transcriptome-wide quelling of the microRNAome?Cancer stem-like cells (CSCs) that possess characteristics associated with normal stem cells (e.g., expression of stem cell markers, the capacity for self-renewal, long term proliferation), but can form tumors (are tumorigenic), have now been described in most tumors. Their tumorigenic functions are especially relevant in the case of aggressive, poorly differentiated, and CSC-rich brain tumors—such as glioblastomas. These tumors are highly heterogeneous in their diverse transcriptomes of cell subpopulations and the whole spectrum of driver mutations, epigenomes, transcriptomes, and phenotypes (7). The current classification of glioblastoma heterogeneity is defined based on the transcriptome of protein-coding genes and consists of three major subtypes: classical, proneural, and mesenchymal (or four in other classification) (8). Tumor-derived, glioblastoma CSCs (GSCs) can also be classified into the same three broad categories in the in vitro culture (9). This picture is muddled though by the temporal evolution of tumors, the co-existence of multiple subtypes within individual tumors, and the multi-directional subtype transitions that happen in response to therapy (8, 10-12). Also, microenvironmental factors (hypoxia, acidity, nutrient flux) and intercellular communication (e.g., by the exchange of extracellular vesicles) further confound glioblastoma's subtype classification (11, 13).

The activation of transcription, although indispensable, is one of many steps during which cells control the processing of genetic information into functional RNAs. Other well-studied steps in the processing of genetic information comprise alternative promoter choice, splicing, editing, and 3′ end formation, all of which end up generating the astonishingly diverse transcriptomes that include the well-regulated and complex output of non-coding RNAs (ncRNAs) from genic and intergenic regions of the genome. The role of these ncRNAs in numerous physiological and pathological processes has been well documented. Numerous classes of functional ncRNAs have been discovered, including microRNAs, long non-coding RNAs, and, most recently, circular RNAs (circRNAs) (14, 15).

Over the years, circRNAs have mostly been disregarded as either transcriptional noise caused by malfunctioning splicing or rare curiosities with no meaningful footprint. A relatively recent demonstration of their widespread and persistent presence within eukaryotic transcriptomes along with numerous examples of cell-type or developmental-stage-specific expression patterns suggestive of regulation has opened new insights into the intricacies of the ncRNA universe, including those of circRNAs that are particularly abundant in the human brain (15, 16). CircRNAs originate from linear transcripts via various mechanisms (15), and their hallmarks are a unique splice junction site, which mediates circularization resulting in a covalently closed “head-to-tail” structure, along with the lack of 5′-3′ polarity and polyadenylated tail, as well as a low probability for encoding protein. CircRNAs have been shown to be long-lived in vivo when compared to their linear counterparts, given that the bulk of RNA turnover involves exonucleases (17). CircRNAs have been shown to act as microRNA sponges (15, 18), but their function remains largely unknown because only a handful contain microRNA target sites. Increasingly, circRNAs are being implicated in numerous cancers; however, the functional relevance of the vast majority is yet to be discovered (19).

While rearrangements of microRNAome in pathologies, including cancer, were observed almost two decades ago, surprisingly little is known on their mechanistic nature. Numerous de-regulations of microRNA expression have been linked to genetic and epigenetic irregularities, altered transcriptional activity, and some missteps in the maturation process. Yet, the overarching explanations of widespread suppression of microRNAome in cancer cells are still lacking. This gap has prompted us to analyze these processes, using a panel of patient-derived, subtype characterized GSCs that are particularly tumorigenic and therapy-resistant as a model, and led to uncovering faulty precursor-to-mature microRNA transition as a widespread circumstance brought about by unusual sub-cellular distribution of DICER complex in the nucleus in these cells. Follow-up scrutiny of nuclear DICER interactome revealed the presence of RNA-protein complex formed by DICER with RNA-binding protein 3 (RBM3) and circular RNA denoted as circ2082. The knowledge about the role of circRNAs in shaping phenotypes of glioblastoma is virtually nonexistent. Thus, we performed the analysis of the circRNAome, which revealed subtype-specific signatures and pointed out to circ2082 as one of the most up-regulated circRNAs in GSCs. The disruption of the discovered complex by circ2082 antisense oligonucleotide led to the cytosolic release of DICER, the resumption of microRNA processing resulting in the restoration of pre-malignant microRNAome, and in consequence, mitigated tumorigenicity in vitro and in vivo. Close inspection of ensuing transcriptome signatures uncovered circRNA/microRNA-dependent effectors that can predict the outcomes of glioblastoma patients. These findings thus revealed the mechanistic foundation of microRNAome de-regulation in the malignant cell and provided new insight into the intricacies of the non-coding RNA universe.

CSC-rich tumors such as glioblastoma are more aggressive than more differentiated ones such as meningioma. Even though differentiation lineage programs exist in CSCs, groups of genes act as brakes to avoid setting these programs in motion. So, releasing those brakes should dampen their aggressiveness (34). Such switch requires sweeping changes in gene expression, so, microRNAs that simultaneously repress multiple factors from certain pathways/developmental programs are efficient regulators of cell fates. Thus it can be argued that microRNA suppression that is common in cancer cells reflects their undifferentiated status (35). As previously shown, the widespread loss of microRNAs in GSCs enhanced their “stemness” (24) and the restoration of pre-malignant microRNA landscape favors more differentiated and a less neoplastic milieu (23). The mechanism of the initial loss of microRNAs is a topic of interest, with a focus on the disruption of microRNA maturation. Yet the mechanisms of cancer cell type-specific alterations of microRNAome are unknown.

As established herein, DICER, an enzyme responsible for microRNA maturation, predominantly localizes in the nucleus of CSCs (as suggested before for breast carcinoma (36)), while in NPCs and other non-malignant cells it is localized in the cytosol (37). Thus, the present study explored the mechanism responsible for this phenomenon and whether this change in localization was accountable for the observed defect in microRNA processing. To this end, nuclear DICER was immunoprecipitated from CSCs for the analysis of its both protein and RNA interactomes. Among proteins bound to nuclear DICER, its canonical interaction partner TRBP2 (38) and RNA-binding protein RBM3 came into view. The interaction with RBM3, which was associated with microRNA biogenesis at the DICER step (26), and with small RNAs of a size corresponding to precursor microRNAs, further supported the idea of impaired microRNA maturation in CSCs as an underlying reason of suppressed microRNAome. Besides these, the presence of the circular RNA circ2082 was also detected. It belongs to the relatively recently discovered non-coding RNA family of circular RNAs implicated in multiple cellular processes (15, 39). Circ2082 originates from the well-established non-coding oncogenic lncRNA MALAT1 that promotes pro-tumorigenic traits in numerous solid tumors, including glioblastoma (28, 40). Tools were used that allowed the present inventors to distinguish between the effects of linear and circular forms of any given transcript. Circularization brings together two sequences that are far apart in a linear molecule, thus creating a unique sequence not found in linear codification, allowing the design of antisense oligonucleotides (ASOs) that exclusively target circular forms. Conversely, antisense targeting linear transcripts remove both forms, as linear structure serves as a template for circular one.

Circ2082 is one of the most highly expressed circRNAs regardless of the GSC subtype, but it is expressed at low levels in NPCs. In contrast, the full-length transcript (MALAT1) from which circ2082 derives is only moderately elevated in GSCs vs. NPCs, suggesting distorted stoichiometry. Circular RNAs are reported to be more stable compared to their parental linear RNAs (17), and without wishing to be bound by theory, it is likely that while cicr2082 accumulates, MALAT1 gets rapidly degraded in cells, accounting for the observed differences. Circularization efficiency may play a role in this discrepancy, as circular transcripts emerge at the expense of linear ones. Thus the cancer cell may promote circularization by yet undetermined mechanisms, directing its transcriptional output toward increased circularization of specific transcripts or the overall enhancement of its circRNAome. At least part of the linear transcript's footprint (either protein-coding or non-coding), both phenotypic and molecular, may be mediated through circRNA originating from such transcript.

More importantly, the discovery of circRNA in the complex with DICER raises the question of its function. Knock-down of strictly nuclear circ2082 shifted the expression of the protein-coding transcriptome in a cell-dependent manner, i.e., knockdown cells remained relatively similar to parental ones. Yet, its effect on the microRNAome was much more pronounced as knockdown cells clustered together but not with their parental cells, indicating far-reaching re-arrangements of their microRNAome. The de-regulation of microRNAs was not random; the ones repressed in CSCs were de-repressed (miR-128 was among the most induced), while few abundant, oncogenic microRNAs (e.g., miR-21) were suppressed. Again, without wishing to be bound by theory, de-repression of a multitude of lowly expressed microRNAs may put a strain on the capacity of processing machinery, thus leading to relative suppression of previously highly expressed microRNAs.

Based on the published association between global downregulation of microRNAs and cancer growth, and without wishing to be bound by theory, it is proposed that the loss of the microRNAome by circ2082-dependent nuclear retention of DICER determines the CSCs' molecular identity and their tumorigenic potential. Thus, targeting of circ2082 reverses the cell fate of CSCs via facilitating the restoration of a pre-malignant microRNAome, and cytosolic release of the DICER complex seems to play a vital role in this phenomenon.

Without wishing to be bound by theory, it is hypothesized that loss of microRNAome by circ2082 dependent DICER nuclear retention determines CSC molecular identity and tumorigenic potential. Thus, the targeting of circ2082 reverses cell fate in CSCs by cytoplasmic re-localization of DICER that mediates microRNAome correction.

Each mature cell that is poised to perform specialized tasks within the human body starts as a primordial cell. So, its journey to cellular adulthood is determined by binary decisions until it reaches its final destination. But when these steps go awry, the stem cell may take a turn down the oncogenic path. To retract the wrong decision made by these primitive cells as they differentiate, it requires unblocking their differentiation potential. So, the modulation of microRNA biogenesis at post-transcriptional steps by non-coding RNA and RNA-binding proteins is the pivotal point of regulatory control over the expression of microRNAs and the cellular processes they affect. However, the extent and conditions under which the microRNA pathway is amenable to regulation at post-transcriptional steps are poorly understood in cancer.

As widespread under-expression of mature microRNAs is often observed in other than brain solid tumors, the restoration of pre-malignant microRNA landscape can be thus highly beneficial in cancer cells regardless of the cell origin. Importantly, circRNA, characterized by a unique circularization site, can be highly accurately targeted using ASO, even in the presence of its linear counterparts, which open possibility for smooth delivery (even systemic, as we showed previously (32)) into the brain.

CircRNAs are recognized as relatively new and promising candidates for biomarkers of pathologies due to their high stability and specificity of detection via junction site. But as protein-coding transcriptome of glioblastoma is well characterized at both bulk and single-cell level, there are no data on the expression of circRNAs in a large cohort of TCGA database. Thus, a transcriptome array was performed in CSCs to assess circ2082-dependent footprint. The data showed that genes differentially deregulated in circ2082 knockdown cells are potent effectors of glioblastoma progression, as reflected by the survival analysis. Analysis of direct, validated targets of selected microRNAs was performed to provide proof that the unblocking of the microRNAome is functionally involved in the survival benefits, evidence for the circ2082-dependent microRNA engagement in this process.

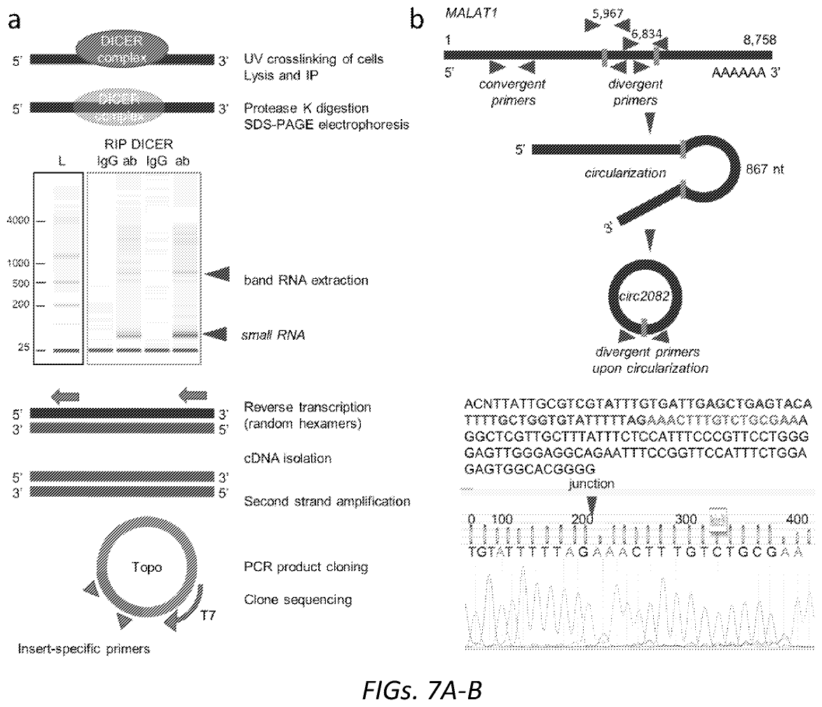

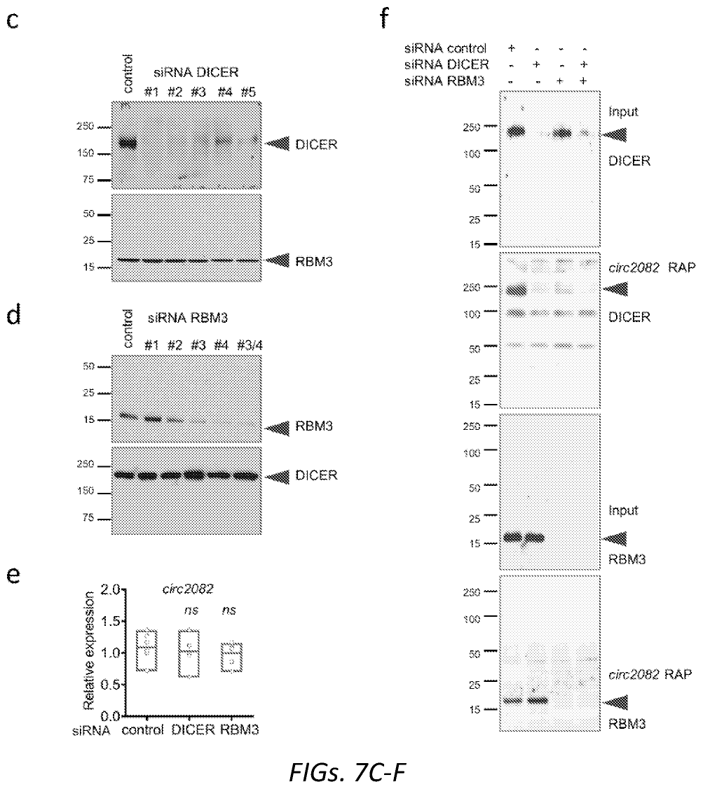

Inhibitory Nucleic Acids Targeting Circ2082 Inhibitory nucleic acids useful in the present methods and compositions include antisense oligonucleotides (ASO), single- or double-stranded RNA interference (RNAi) compounds such as siRNA compounds, modified bases/locked nucleic acids (LNAs), peptide nucleic acids (PNAs), and other oligomeric compounds or oligonucleotide mimetics that hybridize to at least a portion of the target circ2028 nucleic acid comprising the circularization junction (see FIG. 7B and SEQ ID NOs: 35 and 36; the circularization junction site is between nts 57 and 58 of SEQ ID NO:35, and between nts 11 and 12 of SEQ ID NO:36) and modulate its function, i.e., resulting in normalization of DICER localization to the cytosol with re-establishment of microRNAome homeostasis, leading to increased survival time and reductions in tumor growth and/or size.

CIRC28 Circularization Junction Site Target Sequence

(SEQ ID NO: 37)
GTCGTATTTGTGATTGAAGCTGAGTACATTTTGCTGGTGTATTT
TTAG|AAACTTTGTCTGCGA

In SEQ ID NO:37 as shown above, the circularization junction site is between nts 48 and 49 and is indicated by a | symbol. In preferred embodiments, the inhibitory nucleic acids are complementary to a sequence that includes at least 1, 2, 3, 4, 5, 6, or 7 nts from either side of the circularization junction site, i.e., including at least nts 48-49 or 47-50 of SEQ ID NO:37, plus additional nts on one or both sides of the circularization junction site (optionally beyond the length of SEQ ID NO:37, comprising sequences from SEQ ID NO:35 or the human MALAT1 sequence), such that the sequence is long enough to meet the requirements set forth herein of length and to provide a sequence that is unique in the human genome. In some embodiments, the inhibitory nucleic acid comprises at least 10, 11, 12, 13, 14, 15, 16, 17, 18, 19, or all 20 consecutive nucleotides of

(SEQ ID NO: 33)
GTTTCTAAAAATACACCAGC.

In some embodiments, the inhibitory nucleic acids include antisense RNA, antisense DNA, chimeric antisense oligonucleotides, antisense oligonucleotides comprising modified linkages, short interfering RNA (siRNA); a short, hairpin RNA (shRNA); LNA, PNA, or combinations thereof. See, e.g., WO 2010040112.

In some embodiments, the inhibitory nucleic acids are 10 to 50, 10 to 20, 10 to 25, 13 to 50, or 13 to 30 nucleotides in length. One having ordinary skill in the art will appreciate that this embodies inhibitory nucleic acids having complementary portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 consecutive nucleotides in length, or any range therewithin. In some embodiments, the inhibitory nucleic acids are 15 nucleotides in length. In some embodiments, the inhibitory nucleic acids are 12 or 13 to 20, 25, or 30 nucleotides in length. One having ordinary skill in the art will appreciate that this embodies inhibitory nucleic acids having complementary portions of 12, 13, 14, 15, 16, 17, 18, 19, 20, 21, 22, 23, 24, 25, 26, 27, 28, 29 or 30 consecutive nucleotides in length, or any range therewithin (complementary portions refers to those portions of the inhibitory nucleic acids that are complementary to the target sequence).

The inhibitory nucleic acids useful in the present methods are sufficiently complementary to the target RNA, i.e., hybridize sufficiently well and with sufficient specificity, to give the desired effect. “Complementary” refers to the capacity for pairing, through hydrogen bonding, between two sequences comprising naturally or non-naturally occurring bases or analogs thereof. For example, if a base at one position of an inhibitory nucleic acid is capable of hydrogen bonding with a base at the corresponding position of a RNA, then the bases are considered to be complementary to each other at that position. 100% complementarity is not required.

Routine methods can be used to design an inhibitory nucleic acid that binds to the target sequence with sufficient specificity. In some embodiments, the methods include using bioinformatics methods known in the art to identify regions of secondary structure, e.g., one, two, or more stem-loop structures, or pseudoknots, and selecting those regions to target with an inhibitory nucleic acid. For example, “gene walk” methods can be used to optimize the inhibitory activity of the nucleic acid; for example, a series of oligonucleotides of 10-30 nucleotides spanning the length of a target RNA can be prepared, followed by testing for activity. Optionally, gaps, e.g., of 5-10 nucleotides or more, can be left between the target sequences to reduce the number of oligonucleotides synthesized and tested. GC content is preferably between about 30-60%. Contiguous runs of three or more Gs or Cs should be avoided where possible (for example, it may not be possible with very short (e.g., about 9-10 nt) oligonucleotides).

In some embodiments, the inhibitory nucleic acid molecules can be designed to target a specific region of the RNA sequence. For example, a specific functional region can be targeted, e.g., a region comprising a known RNA localization motif (i.e., a region complementary to the target nucleic acid on which the RNA acts). Alternatively or in addition, highly conserved regions can be targeted, e.g., regions identified by aligning sequences from disparate species such as primate (e.g., human) and rodent (e.g., mouse) and looking for regions with high degrees of identity. Percent identity can be determined routinely using basic local alignment search tools (BLAST programs) (Altschul et al., J. Mol. Biol., 1990, 215, 403-410; Zhang and Madden, Genome Res., 1997, 7, 649-656), e.g., using the default parameters.

Once one or more target regions, segments or sites have been identified, e.g., within a target sequence known in the art or provided herein, inhibitory nucleic acid compounds are chosen that are sufficiently complementary to the target, i.e., that hybridize sufficiently well and with sufficient specificity (i.e., do not substantially bind to other non-target RNAs), to give the desired effect.

In the context of this disclosure, hybridization means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. Complementary, as used herein, refers to the capacity for precise pairing between two nucleotides. For example, if a nucleotide at a certain position of an oligonucleotide is capable of hydrogen bonding with a nucleotide at the same position of a RNA molecule, then the inhibitory nucleic acid and the RNA are considered to be complementary to each other at that position. The inhibitory nucleic acids and the RNA are complementary to each other when a sufficient number of corresponding positions in each molecule are occupied by nucleotides which can hydrogen bond with each other. Thus, “specifically hybridisable” and “complementary” are terms which are used to indicate a sufficient degree of complementarity or precise pairing such that stable and specific binding occurs between the inhibitory nucleic acid and the RNA target. For example, if a base at one position of an inhibitory nucleic acid is capable of hydrogen bonding with a base at the corresponding position of a RNA, then the bases are considered to be complementary to each other at that position. 100% complementarity is not required.

It is understood in the art that a complementary nucleic acid sequence need not be 100% complementary to that of its target nucleic acid to be specifically hybridisable. A complementary nucleic acid sequence for purposes of the present methods is specifically hybridisable when binding of the sequence to the target RNA molecule interferes with the normal function of the target RNA to cause a loss of activity, and there is a sufficient degree of complementarity to avoid non-specific binding of the sequence to non-target RNA sequences under conditions in which specific binding is desired, e.g., under physiological conditions in the case of in vivo assays or therapeutic treatment, and in the case of in vitro assays, under conditions in which the assays are performed under suitable conditions of stringency. For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

In general, the inhibitory nucleic acids useful in the methods described herein have at least 80% sequence complementarity to a target region within the target nucleic acid, e.g., 90%, 95%, or 100% sequence complementarity to the target region within an RNA. For example, an antisense compound in which 18 of 20 nucleobases of the antisense oligonucleotide are complementary, and would therefore specifically hybridize, to a target region would represent 90 percent complementarity. Percent complementarity of an inhibitory nucleic acid with a region of a target nucleic acid can be determined routinely using basic local alignment search tools (BLAST programs) (Altschul et al., J. Mol. Biol., 1990, 215, 403-410; Zhang and Madden, Genome Res., 1997, 7, 649-656). Inhibitory nucleic acids that hybridize to an RNA can be identified through routine experimentation. In general the inhibitory nucleic acids must retain specificity for their target, i.e., must not directly bind to, or directly significantly affect expression levels of, transcripts other than the intended target.

For further disclosure regarding inhibitory nucleic acids, please see US2010/0317718 (antisense oligos); US2010/0249052 (double-stranded ribonucleic acid (dsRNA)); US2009/0181914 and US2010/0234451 (LNAs); US2007/0191294 (siRNA analogues); US2008/0249039 (modified siRNA); and WO2010/129746 and WO2010/040112 (inhibitory nucleic acids).

Antisense

In some embodiments, the inhibitory nucleic acids are antisense oligonucleotides. Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under stringent conditions to an RNA. Thus, oligonucleotides are chosen that are sufficiently complementary to the target, i.e., that hybridize sufficiently well and with sufficient specificity, to give the desired effect.

siRNA shRNA

In some embodiments, the nucleic acid sequence that is complementary to a target RNA can be an interfering RNA, including but not limited to a small interfering RNA (“siRNA”) or a small hairpin RNA (“shRNA”). Methods for constructing interfering RNAs are well known in the art. For example, the interfering RNA can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure); the antisense strand comprises nucleotide sequence that is complementary to a nucleotide sequence in a target nucleic acid molecule or a portion thereof (i.e., an undesired gene) and the sense strand comprises nucleotide sequence corresponding to the target nucleic acid sequence or a portion thereof. Alternatively, interfering RNA is assembled from a single oligonucleotide, where the self-complementary sense and antisense regions are linked by means of nucleic acid based or non-nucleic acid-based linker(s). The interfering RNA can be a polynucleotide with a duplex, asymmetric duplex, hairpin or asymmetric hairpin secondary structure, having self-complementary sense and antisense regions, wherein the antisense region comprises a nucleotide sequence that is complementary to nucleotide sequence in a separate target nucleic acid molecule or a portion thereof and the sense region having nucleotide sequence corresponding to the target nucleic acid sequence or a portion thereof. The interfering can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNA interference.

In some embodiments, the interfering RNA coding region encodes a self-complementary RNA molecule having a sense region, an antisense region and a loop region. Such an RNA molecule when expressed desirably forms a “hairpin” structure, and is referred to herein as an “shRNA.” The loop region is generally between about 2 and about 10 nucleotides in length. In some embodiments, the loop region is from about 6 to about 9 nucleotides in length. In some embodiments, the sense region and the antisense region are between about 15 and about 20 nucleotides in length. Following post-transcriptional processing, the small hairpin RNA is converted into a siRNA by a cleavage event mediated by the enzyme Dicer, which is a member of the RNase III family. The siRNA is then capable of inhibiting the expression of a gene with which it shares homology. For details, see Brummelkamp et al., Science 296:550-553, (2002); Lee et al, Nature Biotechnol., 20, 500-505, (2002); Miyagishi and Taira, Nature Biotechnol 20:497-500, (2002); Paddison et al. Genes & Dev. 16:948-958, (2002); Paul, Nature Biotechnol, 20, 505-508, (2002); Sui, Proc. Natl. Acad. Sd. USA, 99(6), 5515-5520, (2002); Yu et al. Proc Natl Acad Sci USA 99:6047-6052, (2002).

The target RNA cleavage reaction guided by siRNAs is highly sequence specific. In general, siRNA containing a nucleotide sequences identical to a portion of the target nucleic acid are preferred for inhibition. However, 100% sequence identity between the siRNA and the target gene is not required to practice the present invention. Thus the invention has the advantage of being able to tolerate sequence variations that might be expected due to genetic mutation, strain polymorphism, or evolutionary divergence. For example, siRNA sequences with insertions, deletions, and single point mutations relative to the target sequence have also been found to be effective for inhibition. Alternatively, siRNA sequences with nucleotide analog substitutions or insertions can be effective for inhibition. In general the siRNAs must retain specificity for their target, i.e., must not directly bind to, or directly significantly affect expression levels of, transcripts other than the intended target.

Modified Inhibitory Nucleic Acids

In some embodiments, the inhibitory nucleic acids used in the methods described herein are modified, e.g., comprise one or more modified bonds or bases. A number of modified bases include phosphorothioate, methylphosphonate, peptide nucleic acids, or locked nucleic acid (LNA) molecules. Some inhibitory nucleic acids are fully modified, while others are chimeric and contain two or more chemically distinct regions, each made up of at least one nucleotide. These inhibitory nucleic acids typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric inhibitory nucleic acids of the invention may be formed as composite structures of two or more oligonucleotides, modified oligonucleotides, oligonucleosides and/or oligonucleotide mimetics as described above. Such compounds have also been referred to in the art as hybrids or gapmers. In some embodiments, the oligonucleotide is a gapmer (contain a central stretch (gap) of DNA monomers sufficiently long to induce RNase H cleavage, flanked by blocks of LNA modified nucleotides; see, e.g., Stanton et al., Nucleic Acid Ther. 2012. 22: 344-359; Nowotny et al., Cell, 121:1005-1016, 2005; Kurreck, European Journal of Biochemistry 270:1628-1644, 2003; FLuiter et al., Mol Biosyst. 5(8):838-43, 2009). In some embodiments, the oligonucleotide is a mixmer (includes alternating short stretches of LNA and DNA; Naguibneva et al., Biomed Pharmacother. 2006 November; 60(9):633-8; Orom et al., Gene. 2006 May 10; 372( ):137-41). Representative United States patents that teach the preparation of such hybrid structures comprise, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference. In some embodiments, DNA/RNA heteroduplexes (HDOs) are used, optionally cholesterol-functionalized by conjugation to cholesterol or α-tocopherol at the 5′ end of the RNA strand are used (see Nagata et al., Nat Biotechnol. 2021 Aug. 12. doi: 10.1038/s41587-021-00972-x). In some embodiments, the modified inhibitory nucleic acid maintains activation of RNase H.

In some embodiments, the inhibitory nucleic acid comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than; 2′-deoxyoligonucleotides against a given target.

A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH2-NH—O—CH2, CH, ˜N(CH3)˜O˜CH2 (known as a methylene(methylimino) or MMI backbone], CH2-O—N(CH3)-CH2, CH2-N(CH3)-N (CH3)-CH2 and O—N(CH3)-CH2-CH2 backbones, wherein the native phosphodiester backbone is represented as O—P—O—CH,); amide backbones (see De Mesmaeker et al. Ace. Chem. Res. 1995, 28:366-374); morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991.

Cyclohexenyl nucleic acid oligonucleotide mimetics are described in Wang et al., J. Am. Chem. Soc., 2000, 122, 8595-8602.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts; see U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

One or more substituted sugar moieties can also be included, e.g., one of the following at the 2′ position: OH, SH, SCH₃, F, OCN, OCH₃ OCH₃, OCH₃ O(CH₂)n CH₃, O(CH₂)n NH₂ or O(CH₂)n CH₃ where n is from 1 to about 10; Ci to C10 lower alkyl, alkoxyalkoxy, substituted lower alkyl, alkaryl or aralkyl; Cl; Br; CN; CF3; OCF3; O-, S-, or N-alkyl; O-, S-, or N-alkenyl; SOCH3; SO2 CH3; ONO2; NO2; N3; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. As one example, the nucleic acid sequence can include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). A preferred modification includes 2′-methoxyethoxy [2′-O—CH₂CH₂OCH₃, also known as 2′-O-(2-methoxyethyl) or (2′-O-MOE)] (Martin et al, HeIv. Chim. Acta, 1995, 78, 486). Other modifications can include 2′-methoxy (2′-O—CH₃), 2′-propoxy (2′-OCH₂ CH₂CH₃) and 2′-fluoro (2′-F). As one example, the nucleic acid sequence can include at least one 2′-O-methyl-modified nucleotide, and in some embodiments, all of the nucleotides include a 2′-O-methyl modification. Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Inhibitory nucleic acids can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine and 2,6-diaminopurine. Kornberg, A., DNA Replication, W. H. Freeman & Co., San Francisco, 1980, pp 75-77; Gebeyehu, G., et al. Nucl. Acids Res. 1987, 15:4513). A “universal” base known in the art, e.g., inosine, can also be included. 5-Me-C substitutions have been shown to increase nucleic acid duplex stability by 0.6-1.2<0>C. (Sanghvi, Y. S., in Crooke, S. T. and Lebleu, B., eds., Antisense Research and Applications, CRC Press, Boca Raton, 1993, pp. 276-278) and are presently preferred base substitutions.

It is not necessary for all positions in a given oligonucleotide to be uniformly modified, and in fact more than one of the aforementioned modifications may be incorporated in a single oligonucleotide or even at within a single nucleoside within an oligonucleotide.

In some embodiments, both a sugar and an internucleoside linkage, i.e., the backbone, of the nucleotide units are modified, e.g., replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds comprise, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

Inhibitory nucleic acids can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

Further, nucleobases comprise those disclosed in U.S. Pat. No. 3,687,808, those disclosed in ‘The Concise Encyclopedia of Polymer Science And Engineering’, pages 858-859, Kroschwitz, J. I., ed. John Wiley & Sons, 1990, those disclosed by Englisch et al., Angewandle Chemie, International Edition’, 1991, 30, page 613, and those disclosed by Sanghvi, Y. S., Chapter 15, Antisense Research and Applications’, pages 289-302, Crooke, S. T. and Lebleu, B. ea., CRC Press, 1993. Certain of these nucleobases are particularly useful for increasing the binding affinity of the oligomeric compounds of the invention. These include 5-substituted pyrimidines, 6-azapyrimidines and N-2, N-6 and O-6 substituted purines, comprising 2-aminopropyladenine, 5-propynyluracil and 5-propynylcytosine. 5-methylcytosine substitutions have been shown to increase nucleic acid duplex stability by 0.6-1.2<0>C (Sanghvi, Y. S., Crooke, S. T. and Lebleu, B., eds, ‘Antisense Research and Applications’, CRC Press, Boca Raton, 1993, pp. 276-278) and are presently preferred base substitutions, even more particularly when combined with 2′-O-methoxyethyl sugar modifications. Modified nucleobases are described in U.S. Pat. Nos. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,596,091; 5,614,617; 5,750,692, and 5,681,941, each of which is herein incorporated by reference.

In some embodiments, the inhibitory nucleic acids are chemically linked to one or more moieties or conjugates that enhance the activity, cellular distribution, cellular uptake, or CNS delivery of the oligonucleotide. Such moieties comprise but are not limited to, lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al, Ann. N. Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Mancharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-t oxycholesterol moiety (Crooke et al., J. Pharmacol. Exp. Ther., 1996, 277, 923-937). See also U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

In some embodiments, the inhibitory nucleic acids are modified to improve delivery to the brain. For example, in some embodiments, the inhibitory nucleic acids are biotinylated or pegylated. For example, 3-biotinylation of phosphodiester (PO)-ASOs or PNAs provides protection against serum and cellular 3′-exonucleases, facilitates conjugation to avidin-based delivery systems (and maintains activation of RNase H) (see Boado et al., J Pharm Sci. 1998 November; 87(11):1308-15).

Locked Nucleic Acids (LNAs)

In some embodiments, the modified inhibitory nucleic acids used in the methods described herein comprise locked nucleic acid (LNA) molecules, e.g., including [alpha]-L-LNAs. LNAs comprise ribonucleic acid analogues wherein the ribose ring is “locked” by a methylene bridge between the 2′-oxygen and the 4′-carbon—i.e., oligonucleotides containing at least one LNA monomer, that is, one 2′-O,4′-C-methylene-β-D-ribofuranosyl nucleotide (see, e.g., Kaupinnen et al., Drug Disc. Today 2(3):287-290 (2005); Koshkin et al., J. Am. Chem. Soc., 120(50):13252-13253 (1998)). LNA bases form standard Watson-Crick base pairs but the locked configuration increases the rate and stability of the basepairing reaction (Jepsen et al., Oligonucleotides, 14, 130-146 (2004)). LNAs also have increased affinity to base pair with RNA as compared to DNA. These properties render LNAs especially useful as probes for fluorescence in situ hybridization (FISH) and comparative genomic hybridization, as knockdown tools for miRNAs, and as antisense oligonucleotides to target mRNAs or other RNAs, e.g., RNAs as described herein.

The LNA molecules can include molecules comprising 10-30, e.g., 12-24, e.g., 12, 13, 14, 15, 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the RNA. The LNA molecules can be chemically synthesized using methods known in the art.

The LNA molecules can be designed using any method known in the art; a number of algorithms are known, and are commercially available (e.g., on the internet, for example at exiqon.com). See, e.g., You et al., Nuc. Acids. Res. 34:e60 (2006); McTigue et al., Biochemistry 43:5388-405 (2004); and Levin et al., Nuc. Acids. Res. 34:e142 (2006). For example, “gene walk” methods, similar to those used to design antisense oligos, can be used to optimize the inhibitory activity of the LNA; for example, a series of oligonucleotides of 10-30 nucleotides spanning the length of a target RNA can be prepared, followed by testing for activity. Optionally, gaps, e.g., of 5-10 nucleotides or more, can be left between the LNAs to reduce the number of oligonucleotides synthesized and tested. GC content is preferably between about 30-60%. General guidelines for designing LNAs are known in the art; for example, LNA sequences will bind very tightly to other LNA sequences, so it is preferable to avoid significant complementarity within an LNA. Contiguous runs of more than four LNA residues, should be avoided where possible (for example, it may not be possible with very short (e.g., about 9-10 nt) oligonucleotides). In some embodiments, the LNAs are xylo-LNAs.

For additional information regarding LNAs see U.S. Pat. Nos. 6,268,490; 6,734,291; 6,770,748; 6,794,499; 7,034,133; 7,053,207; 7,060,809; 7,084,125; and 7,572,582; and U.S. Pre-Grant Pub. Nos. 20100267018; 20100261175; and 20100035968; Koshkin et al. Tetrahedron 54, 3607-3630 (1998); Obika et al. Tetrahedron Lett. 39, 5401-5404 (1998); Jepsen et al., Oligonucleotides 14:130-146 (2004); Kauppinen et al., Drug Disc. Today 2(3):287-290 (2005); and Ponting et al., Cell 136(4):629-641 (2009), and references cited therein.

Making and Using Inhibitory Nucleic Acids

The nucleic acid sequences used to practice the methods described herein, whether RNA, cDNA, genomic DNA, vectors, viruses or hybrids thereof, can be isolated from a variety of sources, genetically engineered, amplified, and/or expressed/generated recombinantly. Recombinant nucleic acid sequences can be individually isolated or cloned and tested for a desired activity. Any recombinant expression system can be used, including e.g. in vitro, bacterial, fungal, mammalian, yeast, insect or plant cell expression systems.

Nucleic acid sequences described herein can be inserted into delivery vectors and expressed from transcription units within the vectors. The recombinant vectors can be DNA plasmids or viral vectors. Expression constructs can include insertion of the nucleic acid sequences in viral vectors, including recombinant retroviruses, adenovirus, adeno-associated virus (AAV), lentivirus, and herpes simplex virus-1, or recombinant bacterial or eukaryotic plasmids. Generation of the vector construct can be accomplished using any suitable genetic engineering techniques well known in the art, including, without limitation, the standard techniques of PCR, oligonucleotide synthesis, restriction endonuclease digestion, ligation, transformation, plasmid purification, and DNA sequencing, for example as described in Sambrook et al. Molecular Cloning: A Laboratory Manual. (1989)), Coffin et al. (Retroviruses. (1997)) and “RNA Viruses: A Practical Approach” (Alan J. Cann, Ed., Oxford University Press, (2000)). As will be apparent to one of ordinary skill in the art, a variety of suitable vectors are available for transferring nucleic acids of the invention into cells. The selection of an appropriate vector to deliver nucleic acids and optimization of the conditions for insertion of the selected expression vector into the cell, are within the scope of one of ordinary skill in the art without the need for undue experimentation. Viral vectors comprise a nucleotide sequence having sequences for the production of recombinant virus in a packaging cell. Viral vectors expressing nucleic acids of the invention can be constructed based on viral backbones including, but not limited to, a retrovirus, lentivirus, adenovirus, adeno-associated virus (AAV), pox virus or alphavirus. The recombinant vectors capable of expressing the nucleic acids of the invention can be delivered as described herein, and persist in target cells (e.g., stable transformants).

Thus in some embodiments, viral vectors, including retrovirus vectors and adeno-associated virus vectors, can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, Blood 76:271 (1990)). A replication defective retrovirus can be packaged into virions, which can be used to infect a target cell through the use of a helper virus by standard techniques. Protocols for producing recombinant retroviruses and for infecting cells in vitro or in vivo with such viruses can be found in Ausubel, et al., eds., Current Protocols in Molecular Biology, Greene Publishing Associates, (1989), Sections 9.10-9.14, and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include ΨCrip, ΨCre, Ψ2 and ΨAm. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. Nos. 4,868,116; 4,980,286; PCT Application WO 89/07136; PCT Application WO 89/02468; PCT Application WO 89/05345; and PCT Application WO 92/07573).

Another viral gene delivery system useful in the present methods utilizes adenovirus-derived vectors. The genome of an adenovirus can be manipulated, such that it encodes and expresses a gene product of interest but is inactivated in terms of its ability to replicate in a normal lytic viral life cycle. See, for example, Berkner et al., BioTechniques 6:616 (1988); Rosenfeld et al., Science 252:431-434 (1991); and Rosenfeld et al., Cell 68:143-155 (1992). Suitable adenoviral vectors derived from the adenovirus strain Ad type 5 d1324 or other strains of adenovirus (e.g., Ad2, Ad3, or Ad7 etc.) are known to those skilled in the art. Recombinant adenoviruses can be advantageous in certain circumstances, in that they are not capable of infecting non-dividing cells and can be used to infect a wide variety of cell types, including epithelial cells (Rosenfeld et al., (1992) supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ, where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham, J. Virol. 57:267 (1986).

Yet another viral vector system useful for delivery of nucleic acids is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro. and Immunol. 158:97-129 (1992). It is also one of the few viruses that may integrate its DNA into non-dividing cells, and exhibits a high frequency of stable integration (see for example Flotte et al., Am. J. Respir. Cell. Mol. Biol. 7:349-356 (1992); Samulski et al., J. Virol. 63:3822-3828 (1989); and McLaughlin et al., J. Virol. 62:1963-1973 (1989). Vectors containing as little as 300 base pairs of AAV can be packaged and can integrate. Space for exogenous DNA is limited to about 4.5 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993).

Nucleic acid sequences used to practice this invention can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Techniques for the manipulation of nucleic acids used to practice this invention, such as, e.g., subcloning, labeling probes (e.g., random-primer labeling using Klenow polymerase, nick translation, amplification), sequencing, hybridization and the like are well described in the scientific and patent literature, see, e.g., Sambrook et al., Molecular Cloning; A Laboratory Manual 3d ed. (2001); Current Protocols in Molecular Biology, Ausubel et al., eds. (John Wiley & Sons, Inc., New York 2010); Kriegler, Gene Transfer and Expression: A Laboratory Manual (1990); Laboratory Techniques In Biochemistry And Molecular Biology: Hybridization With Nucleic Acid Probes, Part I. Theory and Nucleic Acid Preparation, Tijssen, ed. Elsevier, N.Y. (1993).

### Pharmaceutical Compositions

The methods described herein can include the administration of pharmaceutical compositions and formulations comprising inhibitory nucleic acid sequences designed to target a circ2018 RNA as described herein.

In some embodiments, the compositions are formulated with a pharmaceutically acceptable carrier. The pharmaceutical compositions and formulations can be administered parenterally, topically, orally or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration of pharmaceuticals are well described in the scientific and patent literature, see, e.g., Remington: The Science and Practice of Pharmacy, 21st ed., 2005.

The inhibitory nucleic acids can be administered, e.g., as a component of a pharmaceutical formulation (composition). The compounds may be formulated for administration, in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the compositions of the invention include those suitable for intradermal, inhalation, oral/nasal, topical, parenteral, rectal, and/or intravaginal administration. The formulations may conveniently be presented in unit dosage form and may be prepared by any methods well known in the art of pharmacy. The amount of active ingredient (e.g., nucleic acid sequences of this invention) which can be combined with a carrier material to produce a single dosage form will vary depending upon the host being treated, the particular mode of administration, e.g., intradermal or inhalation. The amount of active ingredient which can be combined with a carrier material to produce a single dosage form will generally be that amount of the compound which produces a therapeutic effect, e.g., an antigen specific T cell or humoral response.

Pharmaceutical formulations can be prepared according to any method known to the art for the manufacture of pharmaceuticals. Such drugs can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., nucleic acid sequences of the invention) in admixture with excipients suitable for the manufacture of aqueous suspensions, e.g., for aqueous intradermal injections. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

In some embodiments, oil-based pharmaceuticals are used for administration of nucleic acid sequences as described herein. Oil-based suspensions can be formulated by suspending an active agent in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102.

In addition to viral transfer methods, such as those illustrated above, non-viral methods can also be employed to deliver a nucleic acid compound described herein in the tissue of a subject. Typically non-viral methods of gene transfer rely on normal mechanisms used by mammalian cells for the uptake and intracellular transport of macromolecules. In some embodiments, non-viral gene delivery systems can rely on endocytic pathways for the uptake of the subject gene by the targeted cell. Exemplary gene delivery systems of this type include liposomal derived systems, poly-lysine conjugates, and artificial viral envelopes. Other embodiments include plasmid injection systems such as are described in Meuli et al., J. Invest. Dermatol. 116(1):131-135 (2001); Cohen et al., Gene Ther. 7(22):1896-905 (2000); or Tam et al., Gene Ther. 7(21):1867-74 (2000).

In some embodiments, a nucleic acid as described herein is entrapped in liposomes bearing positive charges on their surface (e.g., lipofectins), which can be tagged with antibodies against cell surface antigens of the target tissue (Mizuno et al., No Shinkei Geka 20:547-551 (1992); PCT publication WO91/06309; Japanese patent application 1047381; and European patent publication EP-A-43075), or using uncharged nanoparticles, e.g., DOPC ([1,2-dioleoyl-sn-glycero-3-phosphatidylcholine])-based NP or DOTAP (lipoplex)-based NPs. The nucleic acids can also be delivered using nanoparticles, e.g., using a nanoparticle delivery system as described in Kapadia et al., J Appl Polym Sci. 2020 Jul. 5; 137(25): 48651 or Sharma et al., Cancer Rep (Hoboken). 2020 October; 3(5): e1271 (e.g., polymersomes, PEG-based micelles, lipoplexes, chitosan-based nanoparticles; lipid/calcium/phosphate (LCP)-based nanoparticles; gold nanoparticles). For example, dendrimer-based delivery systems can also be used, e.g., poly(amidoamine) (PAMAM) and poly(propyleneimine) (PPI) dendrimers, polyglycerol-based dendrimers, polymerized PEG-based dendrimeric core-shell structures, e.g., comprising polyglycerolamine (PG-Amine), polyglyceryl pentaethylenehexamine carbamate, PEI-PAMAM, and/or PEI-gluconolactone, Ionizable lipid nanoparticles (LNPs); see, e.g., Biswas and Torchilin, Pharmaceuticals (Basel). 2013 February; 6(2): 161-183; Schlich et al., Bioeng Transl Med. 2021 May; 6(2): e10213. The inhibitory nucleic acids can be complexed with, conjugated to, adsorbed onto the surface of, or encapsulated or intercalated within any of these delivery agents. See, e.g., Sharma et al., Cancer Rep (Hoboken). 2020 October; 3(5): e1271.

Alternatively, the inhibitory nucleic acids or modified inhibitory nucleic acids can be delivered alone, e.g., “naked.”

Pharmaceutical formulations can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. In alternative embodiments, these injectable oil-in-water emulsions of the invention comprise a paraffin oil, a sorbitan monooleate, an ethoxylated sorbitan monooleate and/or an ethoxylated sorbitan trioleate.

The pharmaceutical compounds can also be administered by in intranasal routes including suppositories, insufflation, powders and aerosol formulations (for examples of steroid inhalants, see e.g., Rohatagi (1995) J. Clin. Pharmacol. 35:1187-1193; Tjwa (1995) Ann. Allergy Asthma Immunol. 75:107-111). Exemplary methods and compositions for brain targeted drug delivery using the nasal route are described in Khan et al., J Control Release. 2017 Dec. 28; 268:364-389. Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In some embodiments, the pharmaceutical compounds can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In some embodiments, the pharmaceutical compounds can be parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

In some embodiments, the pharmaceutical compounds and formulations can be lyophilized. Stable lyophilized formulations comprising an inhibitory nucleic acid can be made by lyophilizing a solution comprising a pharmaceutical of the invention and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. 20040028670.

The compositions and formulations can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587. As used in the present invention, the term “liposome” means a vesicle composed of amphiphilic lipids arranged in a bilayer or bilayers. Liposomes are unilamellar or multilamellar vesicles that have a membrane formed from a lipophilic material and an aqueous interior that contains the composition to be delivered. Cationic liposomes are positively charged liposomes that are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes can also include “sterically stabilized” liposomes, i.e., liposomes comprising one or more specialized lipids. When incorporated into liposomes, these specialized lipids result in liposomes with enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

In some embodiments, the compositions or inhibitory nucleic acids are formulated for delivery to the brain, e.g., cellular delivery systems using conjugates of streptavidin (SA) and monoclonal antibody directed to the transferrin receptor (e.g., OX26) can be used as a carrier for the transport of mono-biotinylated inhibitory nucleic acids such as ASOs or PNAs (see Boado et al., J Pharm Sci. 1998 November; 87(11):1308-15). Glucose-coated polymeric nanocarriers with a particle size of about 45 nm and an adequate glucose-ligand density, which can be bound by glucose transporter-1 (GLUT1) that is expressed on capillary endothelial cells in the brain, can be used for encapsulation of ASOs (see Min et al., Angew. Chem. Int. Ed. 2020, 59, 8173). Other nanocarriers are described in Tsou et al., Small 2017, 13, 1701921; Mendonga et al., Mol. Pharmaceutics 2021, 18, 4, 1491-1506. In some embodiments, cholesterol-functionalized DNA/RNA heteroduplexes that are conjugated to cholesterol or α-tocopherol at the 5′ end of the RNA strand are used (see Nagata et al., Nat Biotechnol. 2021 Aug. 12. doi: 10.1038/s41587-021-00972-x). In some embodiments, the inhibitory nucleic acids are conjugated to clathrin cages or triskelia, e.g., as described in Vitaliano et al., Biological Psychiatry 87(9):S151-S152 (May 2020).

The formulations of the invention can be administered for prophylactic and/or therapeutic treatments. In some embodiments, for therapeutic applications, compositions are administered to a subject who is need of treatment for cancer, or who is at risk of or has cancer as described herein, in an amount sufficient to cure, alleviate or partially arrest the clinical manifestations of the disorder or its complications; this can be called a therapeutically effective amount. For example, in some embodiments, pharmaceutical compositions of the invention are administered in an amount sufficient to decrease tumor size or growth in the subject.

The amount of pharmaceutical composition adequate to accomplish this is a therapeutically effective dose. The dosage schedule and amounts effective for this use, i.e., the dosing regimen, will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; Remington: The Science and Practice of Pharmacy, 21st ed., 2005). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods of the invention are correct and appropriate.

Single or multiple administrations of formulations can be given depending on for example: the dosage and frequency as required and tolerated by the patient, the degree and amount of therapeutic effect generated after each administration (e.g., effect on tumor size or growth), and the like. The formulations should provide a sufficient quantity of active agent to effectively treat, prevent or ameliorate conditions, diseases or symptoms.

In some embodiments, pharmaceutical formulations for oral administration are in a daily amount of between about 1 to 100 or more mg per kilogram of body weight per day. Lower dosages can be used, in contrast to administration orally, into the blood stream, into a body cavity or into a lumen of an organ. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington: The Science and Practice ofPharmacy, 21st ed., 2005.

Various studies have reported successful mammalian dosing using complementary nucleic acid sequences. For example, Esau C., et al., (2006) Cell Metabolism, 3(2):87-98 reported dosing of normal mice with intraperitoneal doses of miR-122 antisense oligonucleotide ranging from 12.5 to 75 mg/kg twice weekly for 4 weeks. The mice appeared healthy and normal at the end of treatment, with no loss of body weight or reduced food intake. Plasma transaminase levels were in the normal range (AST ¾ 45, ALT ¾ 35) for all doses with the exception of the 75 mg/kg dose of miR-122 ASO, which showed a very mild increase in ALT and AST levels. They concluded that 50 mg/kg was an effective, non-toxic dose. Another study by Kratzfeldt J., et al., (2005) Nature 438, 685-689, injected anatgomirs to silence miR-122 in mice using a total dose of 80, 160 or 240 mg per kg body weight. The highest dose resulted in a complete loss of miR-122 signal. In yet another study, locked nucleic acids (“LNAs”) were successfully applied in primates to silence miR-122. Elmen J., et al., (2008) Nature 452, 896-899, report that efficient silencing of miR-122 was achieved in primates by three doses of 10 mg kg-1 LNA-antimiR, leading to a long-lasting and reversible decrease in total plasma cholesterol without any evidence for LNA-associated toxicities or histopathological changes in the study animals.

In some embodiments, the methods described herein can include co-administration with other drugs or pharmaceuticals, e.g., compositions for providing cholesterol homeostasis. For example, the inhibitory nucleic acids can be co-administered with drugs for treating or reducing risk of a disorder described herein.

Methods of Treatment

The methods described herein include methods for the treatment of disorders associated with abnormal apoptotic or differentiative processes, e.g., cellular proliferative disorders or cellular differentiative disorders, e.g., cancer, including both solid tumors and hematopoietic cancers. In some embodiments, the disorder is a solid tumor, e.g., brain, breast, prostate, pancreatic, hepatic, lung, kidney, skin, head and neck, bladder, ovarian, or colon cancer. Generally, the methods include administering a therapeutically effective amount of an inhibitory nucleic acid targeting circ2082, e.g., targeting a circularization junction, as described herein, to a subject who is in need of, or who has been determined to be in need of, such treatment. In some embodiments, the methods include administering a therapeutically effective amount of a treatment comprising a checkpoint inhibitor, a treatment comprising an agent that increases levels of interferons and a checkpoint inhibitor, and/or a standard treatment comprising chemotherapy, radiotherapy, and/or resection.

As used in this context, to “treat” means to ameliorate at least one symptom of the disorder associated with abnormal apoptotic or differentiative processes. For example, a treatment can result in a reduction in tumor size or growth rate, or an increase in likelihood of survival. Administration of a therapeutically effective amount of a compound described herein for the treatment of a condition associated with abnormal apoptotic or differentiative processes will result in a reduction in tumor size or decreased growth rate, a reduction in risk or frequency of reoccurrence, a delay in reoccurrence, a reduction in metastasis, increased survival, and/or decreased morbidity and mortality, inter alia.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include solid tumors, e.g., malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

In some embodiments, the cancer is a brain cancer, e.g., glioma, e.g., astrocytoma, diffuse infiltrating brainstem gliomas (DIPG), oligodendroglioma, optic pathway glioma, or glioblastoma multiforme (GBM).

Additional examples of proliferative disorders include hematopoietic neoplastic disorders, which are typically not solid tumors. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia.

Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit Rev. in Oncol. Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

In some embodiments, the methods include determining expression levels of one or more of DCX, STMN1, MAPT, LCP2, VAMP5, and/or ANXA4 (e.g., in a sample from the subject comprising tumor tissue, e.g., obtained from biopsy, and then using known methods to determine levels, e.g., PCR), and comparing the levels to a reference level. The presence of a level of DCX, STMN1, MAPT, LCP2, VAMP5, or ANXA4 above the reference level indicates that the tumor is likely to be sensitive to a treatment described herein, and the method can further include selecting the subject for treatment and optionally administering a treatment a described herein to the subject.

### EXAMPLES

The invention is further described in the following examples, which do not limit the scope of the invention described in the claims.

Materials and Methods

The following materials and methods were used in the Examples below.

Cell Culture

GSCs and NPC were cultured as neurospheres under stem cell-enriching conditions using neurobasal medium supplemented with 1% glutamine, 2% B27, and 20 ng/mL EGF and FGF (epidermal growth factor and fibroblast growth factor 2, respectively) using ultra-low attachment plates/flasks. The unique identity of cultured patient-derived cells was confirmed by short tandem repeat analysis. All GSCs used in this study are isocitrate dehydrogenase (IDH) wild type (9). Mycoplasma testing was routinely performed by PCR. Mesenchymal and proneural subclass classification by gene expression profiling was described previously (13). Non-malignant brain cells (n=6): including neurons, two NPCs, two astrocytes, brain microvasculature endothelial cells; other cancer cell lines (n=9): two thyroid carcinomas, leukemia, three breast carcinomas, melanoma, two lung carcinomas.

Cell Transfection

Lipofectamine 2000 was used for all transfections. For transfection one μg of plasmid or ten pg of oligo was added in 500 μl of a medium, followed by addition of 6 μl of Lipofectamine 2000 in 500 μl of medium and incubated for 5 min. The two mixtures were pooled and incubated further for 10 min at room temperature. The respective transfection mixture was then added to the 6-well ultra-low attachment plate with 0.5×10⁶ cells. Cells were incubated at 37° C. for 18 h in a 5% CO₂ by 72 h and then harvested by centrifugation (5 min/1000 rpm/4° C.).

Plasmids

cDNA of human RBM3 was cloned into EcoRI and XhoI sites of the pCDH-EF1-copGFP vector.

Cell Cytoplasmic/Nuclear Fractionation

Cytoplasmic and nuclear fractions were isolated via mild lysis and centrifugation using the Nuclear/Cytosol Fractionation Kit (Biovision, Milpitas, CA). Briefly, 2×10⁶ cells were treated with trypsin-EDTA (Gibco) and resuspended with Cytosol Extraction Buffer A. Cytosol Extraction Buffer B were added to the suspension followed by centrifugation to obtain cytosolic protein/RNA containing supernatant whereas the pellet was further processed for nuclear proteins/RNAs.

Protein Purification

Total cell protein content was isolated via extraction for 30 min in ice-cold lysis buffer containing: 50 mM Tris-Cl, pH 7.5, 100 mM NaCl, 1% Triton X-100, 1 mM dithiothreitol (DTT), 1 mM EDTA, 1 mM EGTA, 2 mM Na₃VO₄, 50 mM glycerophosphate, and a protease inhibitor cocktail (GE Healthcare, Piscataway, NJ), followed by centrifugation (15 min/13000 rpm/4° C.).

Quantitative PCR

Total RNA was extracted using a standard Trizol protocol (Invitrogen). The RNA quantity and quality were measured using a NanoDrop 2000 (Thermo Scientific) and analyzed using a Bioanalyzer 2100. Gel images are provided for visualization of fragment sizing and distribution, as well as for a visual representation of the RNA ladder. For RNase R treatment, 1 μg of total RNA was incubated 30 min at 37° C. with or without 2.5 U of RNase R (Epicentre Technologies, Madison, WI). For mRNA analysis, 3 μg total RNA was treated with DNase (Promega) for 2 h to remove genomic DNA. For microRNA analysis, ten ng of total RNA was used. Reverse transcription (RT) was performed using random hexamers and iScript (BioRad), and quantitative PCR (qPCR) was performed using TaqMan or SYBR Green master mix (Applied Biosystems).

Amplification was performed using the StepOnePlus Real-Time PCR System (Applied Biosystems, Foster City, CA), and the software determined Ct thresholds. Expression was quantified using the ΔΔCt method using 18S rRNA (for mRNA/circRNA) or U6 small nuclear RNA (for microRNA or nuclear RNA fraction) as reference genes. PCR products were cloned into pGEM-T Easy, and different clones were picked for Sanger sequencing. Probes and primers are listed in Supplementary Resources Table.

Immunoprecipitation (IP)

Cleared whole cell or cell fraction protein lysates were incubated at 4° C. for three h with the appropriate antibody pre-coupled to protein A/G plus agarose-beads (sc-2003, Santa Cruz Biotechnology). The beads were washed twice with extraction buffer, twice with extraction buffer containing 0.5 M LiCl, and twice with assay buffer (40 mM Tris-Cl, pH 7.5, 0.1 mM EDTA, 5 mM MgCl₂, and 2 mM DTT).

RNA Immunoprecipitation (RIP)

Cells (control) or transfected with pCDH-EF1-copGFP/pCDH-EF1-copGFP-RBM3 vectors were UV cross-linked at 400 mJ/cm2. Cells or nuclei were collected with RIP buffer (100 mM NaCl, 20 mM Tris-Cl (pH 8.0), 0.5 mM EDTA, 0.5% Nonidet P-40, 0.1% Na-deoxycholate, 0.5 mM DTT, 100 U/ml RNasin, protease, and phosphatase inhibitors). One-tenth of each cell or fraction lysate was used for RNA extraction using Trizol reagent (Invitrogen), and the rest was incubated with anti-DICER or IgG or GFP antibodies coupled with Protein A/G Plus Agarose beads (sc-2003, Santa Cruz Biotechnology) overnight at 4° C. Protein/RNA complexes were washed three times with RIP buffer and three times with high-salt buffer (1 M NaCl modified RIP buffer). Samples were then treated with Proteinase K (Invitrogen), and RNA was extracted using Trizol. The qPCR was performed as described above.

RNA-Affinity Purification (RAP)

GSC (2*10⁶) (control or with siRNA mediated knock-down of DICER or RBM3) were UV crosslinked at 400 mJ/cm2, and cell lysates (500 μg) were subjected to pull-down using 3 μg biotin-labeled circ2082 probe (see Table of STAR METHODS) and streptavidin beads at room temperature for 2 h. The reaction was washed three times with RIP buffer and three times with high-salt buffer (1 M NaCl modified RIP buffer). RNA was then digested using RNase A, and bound proteins were analyzed by immunoblotting.

Western Blotting

Proteins were separated by SDS-PAGE, transferred to a polyvinylidene fluoride membrane (Immobilon-P, EMD Millipore) by liquid transfer, and the western blots were probed using the appropriate primary antibodies (1:1000) followed by alkaline phosphatase secondary antibodies (1:5000). The signals were detected using a chemiluminescence system (Thermo Scientific), followed by Gel Dock system (Biorad) imaging.

Single-Molecule Fluorescence In Situ Hybridization (FISH)

Single-molecule FISH was performed on GSCs grown on glass coverslips according to the following protocol. Cells were washed twice in PBS, fixed in 4% paraformaldehyde (Electron Microscopy Sciences) in PBS for 10 min, then washed in PBS and stored in 70% ethanol for ≥2 h at 4° C. Coverslips were equilibrated for ≥2 min in washing buffer (10% formamide, 2×SSC) and probing using custom probes (44) labeled with Alexa Fluor (see Table of STAR METHODS) diluted to 25 nM in hybridization solution (10% formamide, 2×SSC, 100 mg/mL dextran sulfate) in a humidifying chamber at 37° C. overnight. The excess probe was washed for 30 min in washing buffer containing 100 ng/mL DAPI and 5 min in washing buffer to remove the excess of DAPI. Nikon Eclipse Ti microscope was used for signal localization and imaging.

Immunofluorescence

Paraformaldehyde fixed, paraffin-embedded specimens were cut into 5-10 m thick sections and mounted on chromogelatin-precoated slides. After paraffin removal in xylene for 30 min, tissue was hydrated in decreasing grades (98-50%) of ethyl alcohol. Antigen retrieval was achieved by incubation in a sodium citrate buffer (pH 6) and boiled for 20 min. Non-specific antigens were blocked using 10% normal rabbit serum (Jackson ImmunoResearch Laboratories, Inc., West Grove, PA, USA). Incubation with primary antibodies, monoclonal mouse anti-DICER1 (1:150, Thermo Fisher) was performed overnight at 4° C. Afterward, slides were washed in TBS buffer (50 mM Tris-HCl, pH 7.4; 150 mM NaCl) and incubated for one h with secondary antibodies rabbit, anti-mouse conjugated with Alexa Fluor 594 (1:5000, Thermo Scientific). Negative control sections were stained with primary antibodies replaced with either 10% normal rabbit serum (NRS, Abcam) or mouse IgG1 (1:150; Abcam). Finally, sections were mounted in UltraCruz Mounting Medium (Santa Cruz Biotechnology) containing 4′,6-Diamidino-2-Phenylindole (DAPI).

In Vitro Cell Assay

For spheroid formation, GSCs were dissociated to single cells and plated at 500 cells/well in a 96-well plate in 100 μl of supplemented Neurobasal medium. Size and number of spheroids were quantified after 96 h using ImageJ, and spheroid volume was calculated. For limited dilution spheroid assay, single-cell suspensions were plated in ultra-low attachment 96-well plates at different concentrations (from 1 to 500 cells per well) in 0.1 ml of supplemented Neurobasal medium. Cultures were left undisturbed for seven days. After incubation, spheres were imaged using a microscope Nikon Eclipse Ti, the percentage of wells not containing spheres for each cell concentration was calculated and plotted against the number of cells per well.

Human Tissue Processing

The collection of the human operative specimens was performed in accordance with the Brigham and Women's Hospital/Dana Farber Cancer Institute IRB protocol (#10-417) and after obtaining informed consent. Frozen surgical specimens with histopathology-confirmed glioblastoma or normal brains were obtained through the Department of Neuropathology at the Dana Farber Cancer Institute.

Animal Studies

Animal experiments were performed using 6- to 8-week-old immunodeficient athymic mice (FoxN1 nu/nu, Envigo, South Easton, MA), in compliance with all relevant ethical regulations applied to the use of small rodents, and with approval by the Animal Care and Use Committees (IACUC) at the Brigham and Women's Hospital and Harvard Medical School (#2016N000384). For intracranial tumor implantation, a stereotactic frame (Kopf) was used to inoculate each animal in the right striatum with mesenchymal GSC (5000 cells per point, n=3), or GSC admixed with ASO control or circ2082. Mice were euthanized and perfused six days after surgery (for tumor immunohistochemistry and tumor volume analysis) or when they reached their predetermined endpoints (for survival analysis).

RNA Cloning and Sequencing

For the analysis of DICER-bound RNAs, we performed RIP procedure as described above, using anti-DICER and anti-IgG antibodies. Isolated RNA was treated with RNase T1 at a final concentration of 1 U/μl and incubated at 22° C. for 15 min. Reverse transcriptase iScript (BioRad) reactions with random hexamers were followed by TOPO cloning (Invitrogen) and sequencing of clones.

Gene Microarray

Transcriptome expression analysis was performed on total RNA extracted from GSCs (n=10) transfected with ASO control or circ2082. Array Star Inc performed RNA labeling and array hybridization. Briefly, total RNA from each sample was linearly amplified and labeled with Cy3-UTP. The labeled antisense RNAs (cRNAs) were purified using an RNAeasy mini kit (Qiagen). The concentration and specific activity of the labeled cRNAs (pmol Cy3 per μg of cRNA) were measured by a NanoDrop ND-1000. The labeled cRNA (1 μg each) was fragmented by adding 11 μl 10× Blocking Agent and 2.2 μl 25× Fragmentation Buffer, then heated at 60° C. for 30 min and finally, 55 μl 2×GE Hybridization Buffer was added to dilute the labeled cRNA. Hybridization solution (100 μl) was dispensed into the gasket slide and assembled to the gene-expression microarray slide. The slides were incubated for 17 h at 65° C. in an Agilent hybridization oven. Agilent Feature Extraction software (version 11.0.1.1) was used to analyze acquired array images. Quantile normalization and subsequent data processing were performed using the GeneSpring GX v12.1 software (Agilent Technologies).

Circular RNA Microarray

CircRNA expression analysis was performed on total RNA extracted from GSCs (n=10) and NPCs (n=4). Array Star Inc performed RNA labeling and array hybridization. Briefly, total RNA was digested with RNase R (Epicentre, Madison, WI, USA) to remove linear RNA and to enrich circular RNAs. The remaining RNAs were amplified and transcribed into fluorescent cRNA utilizing a random priming method (Arraystar Super RNA Labeling Kit; Arraystar) and hybridized onto the Arraystar circRNA Array (8×15 K, Arraystar) (Rockville, MD, USA). After washing, the arrays were scanned by the Agilent Scanner G2505C. Agilent Feature Extraction software (version 11.0.1.1) was used to analyze acquired array images.

Array Data Analysis

After quantile normalization of the raw data, genes that had flags in at least 3 out of 12 samples as detected (‘All Targets Value’) were chosen for further data analysis. The raw expression intensities were log 2 transformed and normalized by Quantile normalization. Differential analysis between groups was performed by t-test. The cutoffs were p≤0.05 and fold change ≥2.0. Normality was assumed for log 2 transformed normalized intensity values across samples per gene. >90% of genes in our dataset passed the Shapiro-Wilk normality test. Differentially expressed transcripts with statistical significance were identified through volcano plot filtering (GraphPad Prism). Hierarchical clustering was performed using the R software (version 2.15).

nCounter Assay

nCounter in-solution hybridization method (Nanostring) technology and service were used to analyze mature and precursor microRNAs in cells (GSC n=10 and NPC; n=5), glioblastoma tissue specimens (n=10 from 5 donors), and GSC transfected with control or anti circ2082 antisense oligo. Sample preparation and procedure were performed according to the manufacturer's instructions. Briefly, 100 ng of RNA for solution-phase hybridization between the target (mature or precursor) microRNA and reporter-capture probe pairs, excess probes were removed, and the probe/target complexes were aligned and immobilized in the nCounter cartridge (24 samples×800 probes), which was then placed in a digital analyzer for image acquisition and data processing. For data analysis, positive and negative corrections, as well as a sample content normalization to the raw data, were applied as per the manufacturer's guidelines.

Mass Spectrometry-Based Proteomics

Proteomics analysis was performed on immunoprecipitated nuclear and cytoplasmic lysates from GSCs (n=4) with DICER antibodies. The Taplin Mass Spectrometry facility performed mass spectrometry at HMS. Briefly, protein bands were excised from colloidal blue-stained gels (Thermo Fisher Scientific), treated with dithiothreitol and iodoacetamide to alkylate the cysteines before in-gel digestion using modified trypsin (Promega; sequencing grade). The resulting peptides from the whole line were analyzed by nano-liquid chromatography-tandem mass spectrometry (UltiMate 3000 coupled to LTQ-Orbitrap Velos, Thermo Scientific) using a 25-min gradient. Peptides and proteins were identified using Mascot (Matrix Science) and filtered using IRMa software.

TCGA Data Analysis

The collection of the data from TCGA (The Cancer Genome Atlas Research, 2008) was compliant with all applicable laws, regulations, and policies for the protection of human subjects, and necessary ethical approvals were obtained. Experimental and clinical data were downloaded (https://tcga-data.nci.nih.gov/), as described in The Cancer Genome Atlas Research Network. For analysis of mature microRNA, precursor microRNA, and gene expression in glioblastoma, we used normalization of data and aggregation at the feature level as designated by the TCGA glioblastoma the “Level3”. Transcript expression data has been generated from experiments on three different platforms: Affymetrix HT HG U133A (10+488 patient samples×12042 features). Data were analyzed using free available portals (GlioVis; GBM-BioDP; Betastasis) as a resource for accessing and displaying interactive views of TCGA data associated with glioblastoma.

Displaying a summary of experimental data associated with selected genes: The samples (columns on the heatmap) are annotated in two ways: first, according to cluster membership (the optimal number of clusters was determined using NbClust); second, by inspecting the status of a prognostic index (which was computed by weight averaging the gene expressions with the regression coefficients of a multi-gene Cox proportional hazards model). The gene names are annotated with their respective Hazard Ratios in a multi-gene Cox proportional hazards model. When search results involve more than 50 genes, we filter them by keeping the 50 genes whose expression is the most varied among the samples.

Performing gene survival analysis: The Kaplan-Meier survival curve compare samples stratified according to gene expression levels. The default options stratified samples into two groups: those with expression levels smaller than the median over the subgroup, and those with expression levels higher than the median. For gene searches that result in multiple hits, we analyzed how the expression profiles impact survival. We performed two types of survival analyses: first, the optimum clusters were selected by the stratification of the samples according to the heatmap cluster membership (see the first annotation bar), where the optimal number of clusters is picked out algorithmically using silhouette width index. Next, we used a Kaplan-Meier model to analyze the differences in survival between groups using a log-rank statistic. These analyses were performed using the “NbClust” package in R.

Displaying heatmap clustering of gene and miRNA expression data correlation: For selected multiple hits of miRNAs, we present a heatmap of the correlation between the expression of genes and microRNAs. Each cell of the heatmap represents how the expression of the gene in the row, and the microRNA in that column are correlated, and it is annotated with the correlation value. A pairwise Pearson correlation analysis was performed for the selected six microRNAs. The results were displayed as a heat map using hierarchical clustering analysis using the average linkage distance metric.

Quantification and Statistical Analysis

Graphs (scatters plot, box plots, PCA) were generated, and statistical analyses were performed using GraphPad Prism 7. Statistical parameters, including the value of n, statistical test, and statistical significance (p-value), are reported in the figures and their legends. For studies involving mouse tissues, replicates refer to samples derived from different mice. For studies involving cell culture, replicates refer to technical (transfection) or biological (cells/tissues obtained from a different patient) replicates. No statistical methods were used to predetermine the sample size. Statistical tests were selected based on the desired comparison. Unpaired two-tailed t-tests were used to assess significance when comparing data between two variances. One-way ANOVA was used to determine significance when comparing data between ≥3 variances; significant ANOVA results were followed by post hoc testing either comparing every mean with every other mean (Tukey's multiple-comparison test) or comparing every mean to the wild-type mean (Dunnett's multiple-comparison test). For the differential expression of global measurements (platforms), the DESeq2 software-generated adjusted p values using the Wald test with the Benjamini-Hochberg procedure to correct for multiple hypothesis testing. The Mann-Whitney test was used to compare cumulative distributions of gene fold changes between two gene sets.

### Reagents and Resources Used.

| | | |
|---|---|---|
| REAGENT or RESOURCE | SOURCE | IDENTIFIER |

Antibodies

| | | |
|---|---|---|
| DICER (D38E7) Rabbit | Cell Signaling | mAb #5362 |
| | Technology | |
| DICER Rabbit | Thermo Fisher | PA5-23088 |
| TRBP2 (D7C8K) Rabbit | Cell Signaling | mAb #62043 |
| | Technology | |
| RBM3 Rabbit | Invitrogen | PA5-51976 |
| HISTONE H3 (D1H2) XP ® Rabbit | Cell Signaling | mAb #4499 |
| | Technology | |
| TUBULINα Mouse (clone DM1A) | Sigma-Aldrich | T9026 |
| GFP Mouse IgG2A Clone # 454518 | R&D Systems | MAB4240 |
| Secondary Rabbit anti-Mouse monoclonal | Novus Biologicals | MAB0033 |
| Secondary Goat anti-Rabbit polyclonal | Thermo Fisher | 32460 |

Bacterial and Virus Strains

| | | |
|---|---|---|
| BL21 Star (DE3) Chemically Competent | Thermo Fisher | C601003 |
| E. coli* | | |

Biological Samples

| | | |
|---|---|---|
| Patient brain tissue | 45 | N/A |
| Patient-derived GSCs | 13 | N/A |
| NPC cells | Millipore Sigma | SCC007 |
| | Lonza 46 | PT-2599 |
| | | N/A |

Chemicals, Peptides, and Recombinant Proteins

| | | |
|---|---|---|
| EGF | Pepro Tech | AF-100-15 |
| FGF | Pepro Tech | 100-18C |
| B27 | Gibco | 21103049 |
| Neurobasal | Gibco | 21103049 |
| TRIzol ™ Reagent | TermoFisher | 15596018 |
| RNase R | Epicentre | RNR07250 |

Critical Commercial Assays

| | | |
|---|---|---|
| Power Up SYBR Green Master Mix | Thermo Fisher | A25743 |
| TaqMan Fast Advanced Master Mix | Thermo Fisher | 4444963 |
| Human v3 miRNA Assay | Nanostring | CSO-MIR3-12 |
| Human custom pre-miR Assay | Nanostring | This paper |
| Agilent whole human genome microarray | Arraystar | Agilent-026652 4x44K |
| | | v2 |
| Arraystar Human Circular RNA Array | Arraystar | AS-S-CR-H-V2.0 |
| TaqMan microRNA Assay | Applied Biosystems | 4427975 |
| TaqMan Pre-miRNA Assays | Applied Biosystems | 4426961 |
| iScript ™ cDNA Synthesis Kit | Bio-Rad | 1708890 |
| Nuclear/Cytosol Fractionation Kit | Bio Vision | K266 |
| Lipofectamine 2000 | Invitrogen | 11668030 |
| Genome microarray | This application | Deposition: GSE146440 |
| Circular RNA Array | This application | Deposition: GSE146463 |

Experimental Models: Organisms/Strains

| | | |
|---|---|---|
| Mouse: athymic | Envigo | code#069 |

Recombinant DNA

| | | |
|---|---|---|
| pCDH-EF1-copGFP vector | System Biosciences | CD511B-1 |
| pCDH-EF1-copGFP-RBM3 | This application | N/A |

Software and Algorithms

| | | |
|---|---|---|
| ImageJ | 47 | imagej.nih.gov |
| Glio Vis | 48 | gliovis.bioinfo.cnio.es/ |
| | | ?ref=labworm |
| GBM-BioDP | 49 | gbm-biodp.nci.nih.gov |
| Betastasis | 50 | betastasis.com |
| Subcell Barcode | 51 | subcellbarcode.org |

Other

| | | |
|---|---|---|
| TCGA glioblastoma platforms data set: | 52 | cancer.gov/about- |
| Affymetrix HT HG U133A | | nci/organization/ccg/ |
| Agilent Human miRNA 8x15K | | research/structural- |
| Affymetrix Human Exon 1.0 ST | | genomics/tcga |

Oligonucleotides

| | | | SEQ ID |
|---|---|---|---|
| Sequence | Source | Identifier | NO: |
| siRNA targeting sequence: DICER | QIAGEN | | |
| #1 AATGTGCTATCTGGATCCTAG | | SI00300006 | 1 |
| #2 CTGCTCGAAATCTTACGCAAA | | SI04753987 | 2 |
| #3 AAGGACGGTGTTCTTGGTCAA | | SI02645993 | 3 |
| #4 CTCGCATAGGCATTCCCAGTA | | SI02645986 | 4 |
| #5 ATCGATCCTATGTTCAATCTA | | SI02645972 | 5 |
| siRNA targeting sequence: RBM3 | QIAGEN | | |
| #1 CAGATCCGTGTGGATCATGCA | | SI04297755 | 6 |
| #2 TGGGACGTTTGTAGAACCTGA | | SI04296166 | 7 |
| #3 ACCGACGAGCAGGCACTGGAA | | SI04261215 | 8 |
| #4 CAAGGAATAATTTCTGATCCA | | SI04146821 | 9 |
| siRNA control: | QIAGEN | 1027280 | |
| QPCR primers: | Invitrogen | N/A | |
| MALATI (linear, convergent) | | | 10 |
| F: TGATAGCCAAATTGAGACAA | | | |
| R: TTCAGGGTGAGGAAGTAAAA | | | 11 |
| circ2082 (circular, divergent) | | | |
| F: GAAGGAAAAAATCCAGCTGA | | | 12 |
| R: GAATAAAATTTGTCTTTCCTGCC | | | 13 |
| circ2082 (circular, convergent) | | | |
| F: AGGATTTGAGCGGAAGAACGA | | | 14 |
| R: ACACCAGCAAAATGTACTCAGC | | | 15 |
| GLI2 | | | |
| F: CTGTGGGTTAGGGATGGACTG | | | 16 |
| R: GTAAAGTGGGTGGACGTTGCA | | | 17 |
| JARID | | | |
| F: GACACCAAACCCAATCACCAC | | | 18 |
| R: GTTCAACCTGCCACTGACCTT | | | 19 |
| CCNG1 | | | |
| F: CCTTCTGTGTTGGCATTGTCTATC | | | 20 |
| R: CAAGCTCTTGCCAGAAGGTCAG | | | 21 |
| SART3 | | | |
| F: GGAGATTTGGCAGGCATACCTTG | | | 22 |
| R: CTCTTCCACCTCCTGCTTCAGA | | | 23 |
| MET | | | |
| F: TAGCCAACCGAGAGACAAGC | | | 24 |
| R: TGTGCTCCCACCACTAATAAAAG | | | 25 |
| PDCD4 | | | |
| F: TGTAAACCCTGCAGATCCTGA | | | 26 |
| R: TGGAGGATGCTGAAATCCAAT | | | 27 |
| 18S rRNA | | | |
| F: AACTTTCGATGGTAGTCGCCG | | | 28 |
| R: CCTTGGATGTGGTAGCCGTTT | | | 29 |
| RBM3 cloning primers: | Invitrogen | Herein | |
| F: AAAGAATTCATGTCCTCTGAAGAAGGA, | | | 30 |
| R: AAACTCGAGTCAGTTGTCATAATTGTC | | | 31 |
| Antisense oligo (ASO): | | Herein | |
| Control: GCGTATTATAGCCGATTAAC | | | 32 |
| circ2082: GTTTCTAAAAATACACCAGC | | | 33 |
| Knockdown and in situ visualization: | | | |
| Phosphorothioate bond-Cy5 | IDT | | |
| RAP assay: | | | |
| 5' biotinylated | Invitrogen | | |
| FISH assay: | Thermo | | |
| CCATTAAAGAGTGTTCGCAGACAAA | Fisher | | |
| GTTTCTAAAAATACACCAGCAAAATGTAC | | | 34 |
| TaqMan microRNA probes: | Life | Assay ID: | |
| miR-128 | Technologies | 002216 | |
| miR-124-3p | | 003188 | |
| miR-1 | | 002222 | |
| miR-10b-5p | | 002218 | |
| miR-21-5p | | 000397 | |
| miR-31-5p | | 002279 | |
| U6 | | 001973 | |
| TaqMan pre-microRNA probes: | QIAGEN | Assay ID: | |
| MIR128-1 | | MP00000574 | |
| MIR128-2 | | MP00000588 | |
| MIR124-1 | | MP00000371 | |
| MIR124-2 | | MP00000378 | |
| MIR124-3 | | MP00000392 | |
| MIR1-1 | | MP00003990 | |
| MIR1-2 | | MP00004053 | |
| MIR10-2 | | MP00000168 | |
| MIR21 | | MP00001498 | |
| MIR31 | | MP00007889 | |

### Example 1. The Expression of the Glioblastoma microRNAome is Suppressed

Our efforts initially focused on assessing the extent of microRNAome re-arrangements in several settings relevant to glioblastoma. These included analysis of (i) The Cancer Genome Atlas (TCGA) database, (ii) collected tumor tissue paired with matching (i.e., collected from the same individual), adjacent brain tissue devoid of gross pathology, and (iii) the selection of subtype-characterized GSCs and non-malignant neural progenitor/stem cells (NPCs). As an additional dimension of this global analysis, we selected several microRNAs that have been identified in glioblastoma as either well-known tumor suppressors and oncogenes or as subtype-predictive and scrutinized the expression of their both precursor and mature forms. These include miR-124 and miR-1 (low expression in all GSCs (20) (21)), and miR-128 (particularly low expression in the mesenchymal GSC subtype (11), as well as pan-glioblastoma highly expressed miR-21 (22), and miR-10b highly expressed in the proneural GSC subtype (23), and the mesenchymal GSC-specific miR-31 (24).). As these microRNAs are well characterized on the transcriptional level, the phenotypic consequences of their de-regulation are well-defined, and their mRNA targets are convincingly verified, they can serve as indices for levels of expression in glioblastoma.

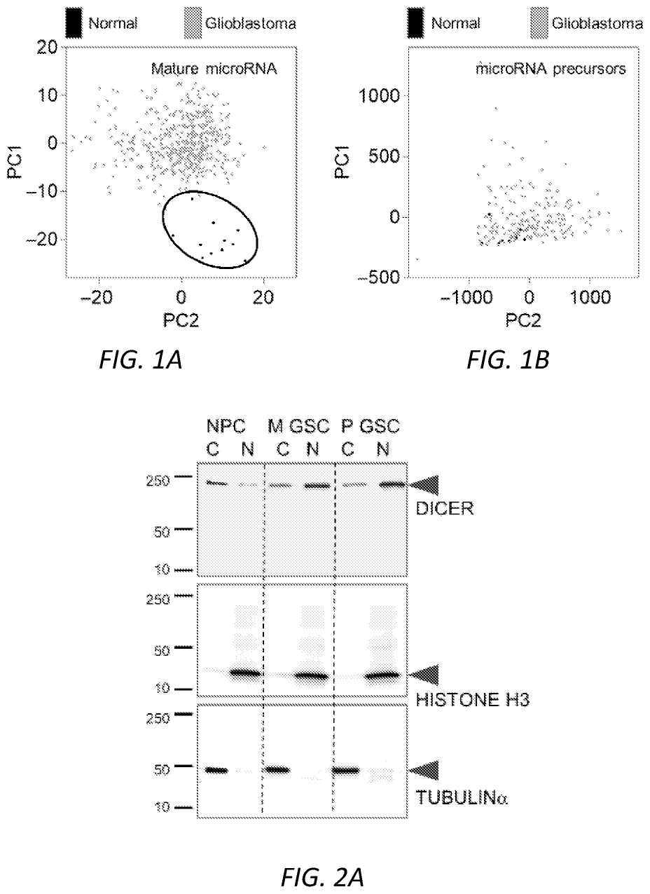

Analysis of all these datasets revealed vastly different but consistently suppressed expression of the mature microRNAome in glioblastoma tissue/GSCs when compared to brain/NPCs, with some subtype-specific distribution in GSCs. Also as expected, the expression of tumor suppressor microRNAs was low and oncogenic microRNA high in tumor/tumor-derived cells, with some distinct subtype characteristics (e.g., miR-128 was particularly low in the mesenchymal (M) GSCs, while miR-10b and miR-31 were elevated in the proneural (P) and M GSCs, respectively). However, levels of selected precursors did not display significantly different patterns between malignant and non-malignant material. Global principal component analysis in the TCGA glioblastoma dataset confirmed the clear separation between groups using mature microRNAs, but not their precursors (FIGS. 1A-B). These findings were thus the first clue that the de-regulation of microRNA processing/maturation machinery may indeed be the primary culprit for the observed widespread suppression of the glioblastoma microRNAome.

### Example 2. DICER Localizes to the Nucleus in Glioblastoma Cells Instead of the Cytosol

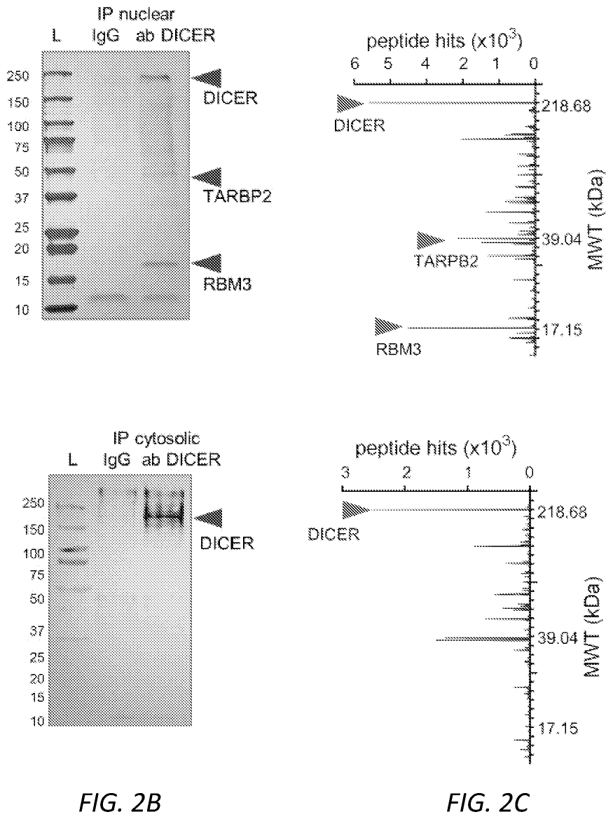

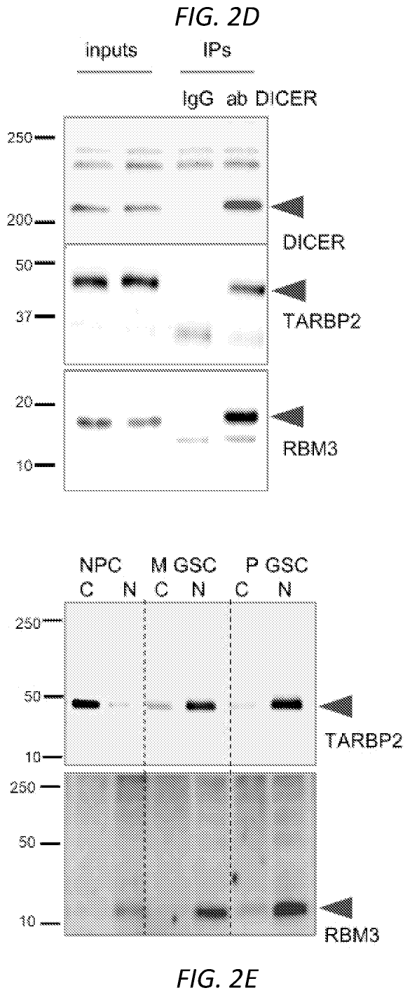

To explore this further, we analyzed the expression of genes/proteins forming the major enzymatic complexes responsible for the transition between primary, precursor, and mature forms of microRNA—DROSHA and DICER. Several proteins compose the enzymatic core of DICER complex, with the most crucial co-factor of DICER being RISC-loading complex subunit TAR (HIV) RNA-Binding Protein 2 (TARBP2), which increases the rate of RNA substrate recognition by DICER and the stability of DICER/RNA substrate complexes (25). Analysis of the TCGA database showed no significant difference in the expression of DROSHA and DICER between a normal brain and glioblastoma, but the expression of TARBP2 was markedly elevated in tumor tissue. However, none of these genes/proteins correlated with the survival of glioblastoma patients, suggesting that differences in their expression levels are unlikely to be that relevant to glioblastoma outcome. However, database querying (subcellbarcode.org) suggested that in cancer cell lines (including glioblastoma), DICER and TARBP2 are mostly nuclear, while the canonical DICER step involving the maturation of microRNA takes place in the cytosol. Indeed, when we compared the distribution of DICER across the cellular compartments of GSCs and NPCs the difference became apparent: while in NPCs DICER was, as expected, predominantly cytosolic (less than 20% in the nucleus), in M and P GSCs it was decidedly nuclear (70-80% based on densitometry analysis) (FIG. 2A). To confirm this distribution difference in vivo, we analyzed the subcellular distribution of DICER in GSC-originated xenografts in mice brains. DICER was indeed predominantly nuclear in GSCs (GFP-positive), while in cells from surrounding tissue (GFP-negative), it was primarily cytosolic. These results clearly demonstrated that the change in subcellular localization of DICER is particular to GSCs.

### Example 3. RBM3 is a Novel Interacting Partner of Nuclear but not Cytosolic DICER

The above finding prompted us to elucidate the mechanism for this change in subcellular localization by analyzing the interactome of nuclear DICER. Immunoprecipitation of DICER from the nuclear fraction of GSCs allowed the identification of interacting proteins and RNAs. Among proteins in the immunoprecipitate, we identified DICER, and its canonical interaction partner TARBP2 (25). In addition, RNA binding motif protein 3 (RBM3) was found to be the most significant nuclear DICER-interacting protein, but interestingly no binding was detected between cytosolic DICER and RBM3 or TARBP2 (FIGS. 2B-C). RBM3 is a highly conserved protein engaged in the biosynthesis of different RNA species (including microRNAs (26)), and it is believed to function as a proto-oncogene associated with tumor progression and metastasis (27). Although RBM3 was strongly expressed in glioblastoma, its expression did not correlate with patient survival. All these interactions were further confirmed by immunoprecipitation and Western blotting (FIG. 2D). Additionally, the subcellular distribution of TARBP2 mirrors that of DICER and RBM3 is strictly nuclear as expected from the data query (FIG. 2E). These findings thus suggested that RBM3 is a novel protein interacting partner of the nuclear, but not cytosolic DICER complex.

### Example 4. Circ2082, a circRNA that is Highly Expressed in Cancer, Binds to RMB3 and is Part of the Nuclear DICER Complex

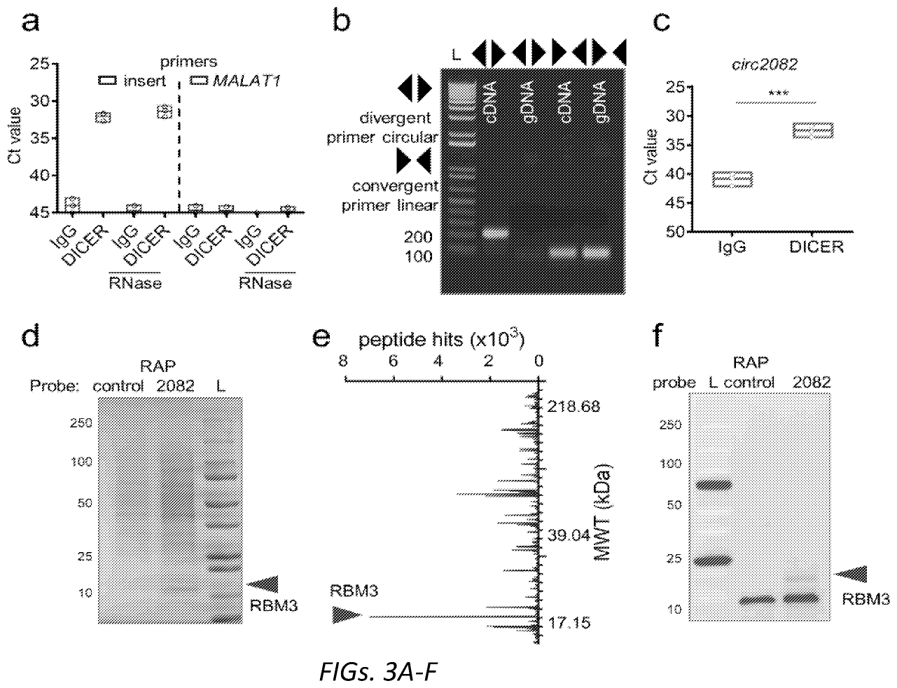

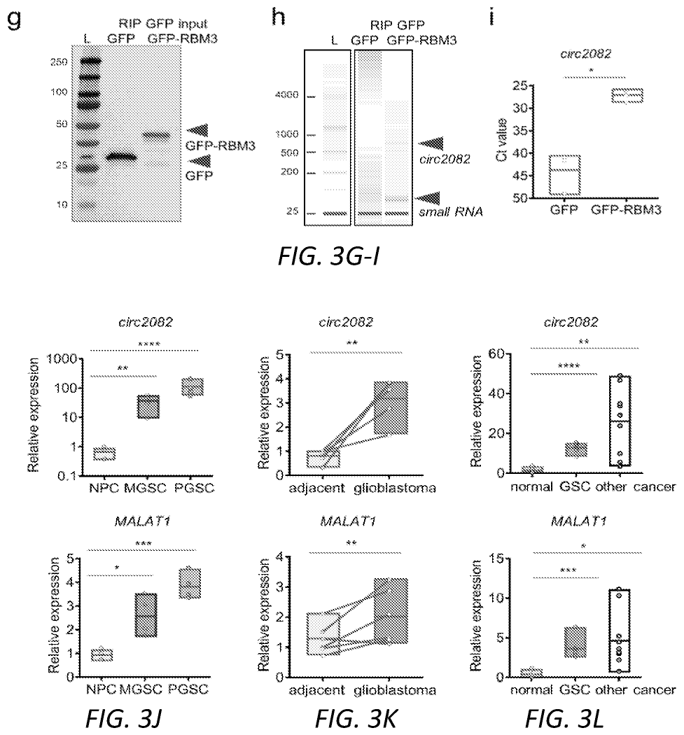

Having discovered RBM3 as a novel protein binding to nuclear DICER, we then analyzed nuclear DICER's RNA interactome. We detected a population of small RNAs whose size corresponded to that of precursor microRNAs, and one prominent, specific band (FIG. 7A). Sequencing of the cDNA obtained from the immunoprecipitate revealed that it was a fragment located close to the 3′-end of MALAT1, a long ncRNA with well-recognized pro-tumorigenic function in multiple malignancies, including glioblastoma (28). Although quantitative polymerase chain reaction (qPCR) confirmed the enrichment of identified fragment in a nuclear immunoprecipitate of DICER, this approach failed to detect a larger fragment corresponding to MALAT1 (FIG. 3A). This, and the fact that the PCR product did not disappear upon RNase treatment (FIG. 3A) led us to the hypothesis that this fragment may be a circular RNA (circRNA) originating from the MALAT1 transcript. Circularization-predicting software (http://www.circbase.org) indeed revealed that there was a high probability for a circRNA arising from this region of linear MALAT1 (FIG. 7B, top). Based on this, we designed both divergent and convergent primers to distinguish between linear and circular forms of this transcript and to detect the junction site (FIG. 3B, FIG. 7B, bottom). CircBase annotated this circRNA as hsa_circ_0002082 (chr11:65271199-65272066), and we abbreviated it as circ2082. Using circular-specific primers, we demonstrated significant enrichment of circ2082 in the nuclear DICER immunoprecipitate (FIG. 3C). To test the interaction between circ2082 and its protein partners, we used RNA antisense purification (RAP) assay followed by mass-spectrometry and confirmed the presence of RBM3 in the circ2082 complex (FIGS. 3d-F). In a set of reciprocal approach experiments, we used a fusion GFP-RBM3 protein to circumvent the lack of immunoprecipitation-grade RBM3 antibodies (FIG. 3G), and we detected circ2082 in the GFP immunoprecipitate (FIG. 3H-I). Finally, we performed a series of circ2082 protein partners knock-down experiments (FIGS. 7C-D) followed by RAP assay to assess the inter-dependency of proteins as binding partners to circ2082. Importantly, the knockdown of neither protein affected the levels of circ2082 (FIG. 7E). While RBM3 was readily detectable in pull-down material regardless of the presence of DICER, the DICER itself was present only with RBM3 intact (FIG. 7F), suggesting that only RBM3 binds directly to circ2082. These results provided proof for the existence of nuclear RNA/protein complex consisting of proteins indispensable in microRNA maturation (DICER), RNA biogenesis/processing (RBM3), and a non-coding, circular RNA (circ2082) originating from notorious non-coding oncogene—MALAT1.

This discovery prompted us to take a closer look at the circRNAome of GSCs and NPCs. To this end, we used the Human Circular RNA Array. We detected 12,659 out of 13,617 probes on Arraystar human circular RNA array (confirming their abundance in the human brain (16)) in both types of cells. Of these, 478 were significantly down-regulated in GSCs while almost twice as many—792 were significantly up-regulated. Circ2082 was among the top circRNAs upregulated in GSCs. The analysis of the expression of circ2082 and its transcript of origin—MALAT1, revealed several interesting details. Firstly, although both transcripts were high in GSCs, and neither of them was subtype-specific, circ2082 was more profoundly up-regulated in GSCs than its linear parental transcript (FIG. 3J, ˜50-100-fold vs. ˜3-fold). Secondly, the degree of overexpression of circ2082 in glioblastoma in comparison to adjacent matched brain tissue was again higher than that of linear MALAT1 (FIG. 3K). Finally, both transcripts were elevated to various degrees in cancers other than glioblastoma, again more strongly in the case of circ2082 (FIG. 3L).

As glioblastoma is notorious for its invasiveness, we cannot exclude the possibility that seemingly healthy tissue can contain a fraction of tumor cells. Yet the compromised purity of the sample can be perceived as somewhat ambiguous as the stromal component often infiltrates a bulk tumor tissue. Also, notably, the matching pairs of samples were harvested from the same individual, effectively nullifying high patient-to-patient heterogeneity. Finally, as both circ2082 and MALAT1 are significantly lower in the normal brain, it implies that the significance detected by us would only become stronger if we have dealt with pure populations. Indeed, cell assays using cancer and non-malignant cells substantiated our bulk tissue findings. These results strongly suggested that while both MALAT1 and its circularized fragment, circ2082, are overexpressed in malignant cells, the circular form is, in most cases, present at higher levels.

### Example 5. Circ2082 Knock-Down Leads to Normalization of DICER Localization to the Cytosol with Re-Establishment of microRNAome Homeostasis

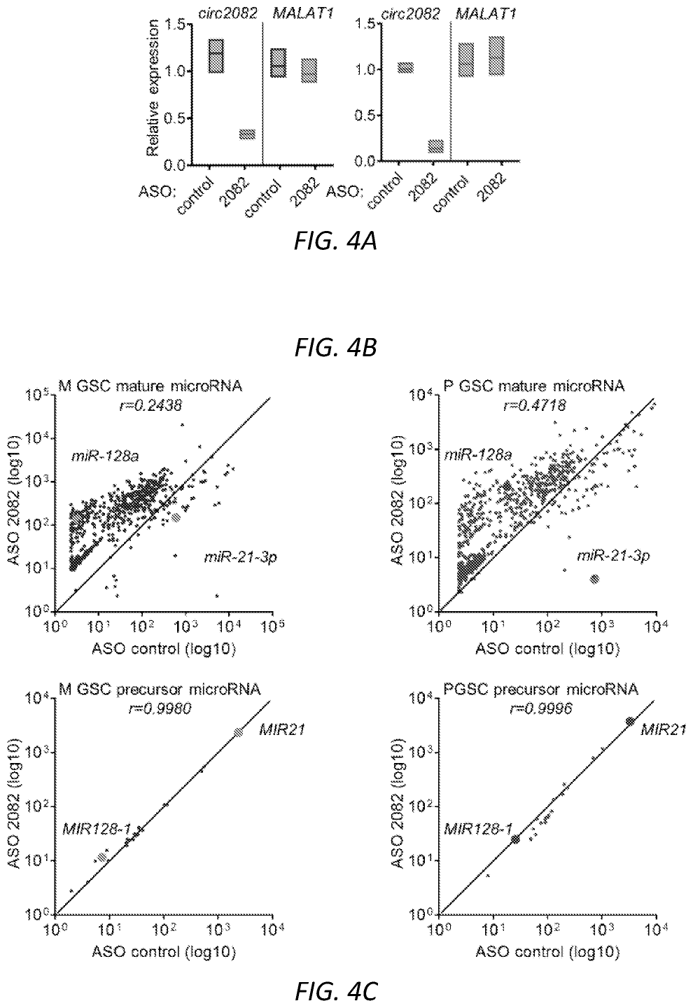

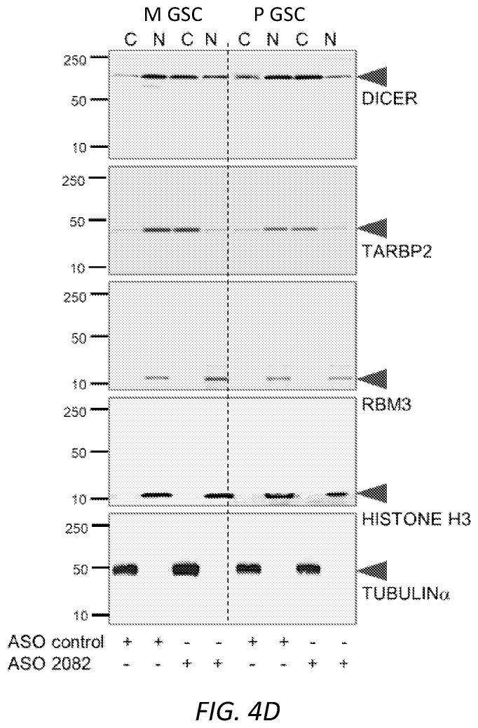

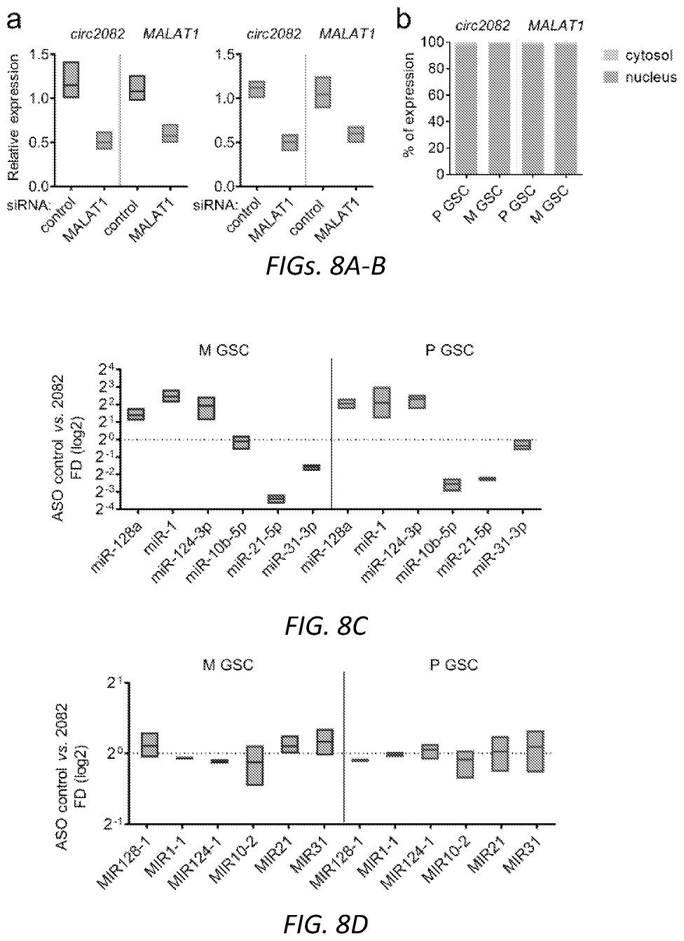

Circularization generates a unique sequence with no homology in the entire human genome, allowing precise targeting of circ2082 via antisense oligonucleotide (ASO), which leaves the linear parental transcript intact (FIG. 4A). Conversely, siRNA-mediated knock-down of MALAT1 effectively removes both linear and circular transcripts, as the former is the source of origin for the latter (FIG. 8A). Importantly, both transcripts are almost exclusively nuclear (FIG. 8B), which we already demonstrated for linear MALAT1 (13). Having the ability to silence circ2082 effectively and specifically, we attempted to characterize the molecular and phenotypic footprint of the knockdown. To this end, we analyzed changes to the protein-coding transcriptome and microRNAome in circ2082 knockdown GSCs. Strikingly, the effect of circ2082 knockdown on the protein-coding transcriptome, albeit significant, was cell-type-dependent. Conversely, the impact of circ2082 expression on the microRNAome was so potent that knockdown cells from both subtypes clustered together, apart from their parental cells. Detailed analysis of microRNAome effect revealed the pattern that was common for both subtypes: widespread de-repression of numerous weakly expressed microRNAs and inhibition of a relatively few that were strongly expressed in control GSCs (FIG. 4B). The analysis of specific microRNAs with known function or subtype-predictive ones (same as in FIGS. 1A-B) confirmed this—the expression of tumor-suppressive miR-128, miR-124, and miR-1 was unblocked regardless of subtype. Simultaneously, the expression of oncogenic miR-21, P GSC-specific, oncogenic miR-10b, and M GSC-specific miR-31 were suppressed (FIG. 8C). Importantly the levels of their precursors were not altered upon circ2082 knockdown (FIG. 4C, 8D), suggesting again that unblocked processing rather than transcriptional change was responsible for the observed phenomenon. Finally, we were able to demonstrate that knockdown of circ2082 resulted in cytosolic re-localization of the DICER complex, while RBM3 was and remained strictly nuclear (FIG. 4D). These results showed that nuclear retention of the DICER complex, a molecular consequence of high levels of circ2082 in GSCs, leads to the blockade of mature microRNAome.

### Example 6. Circ2082 Knock-Down has Anticancer Effects

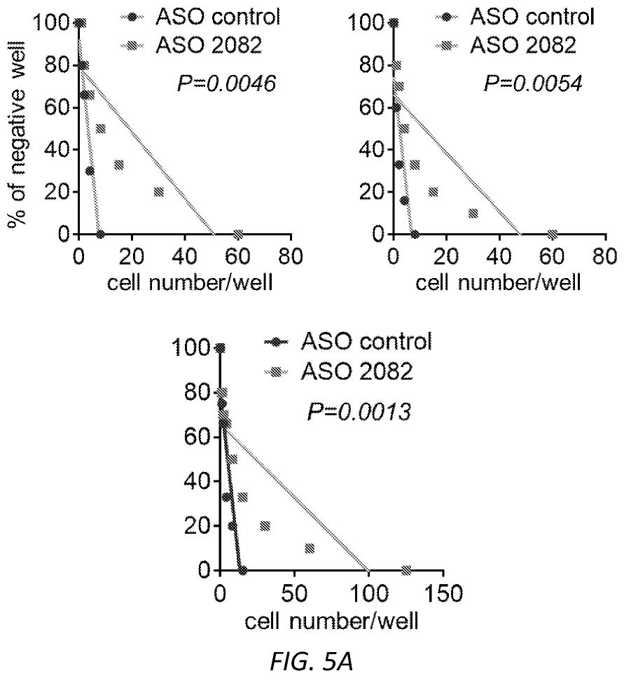

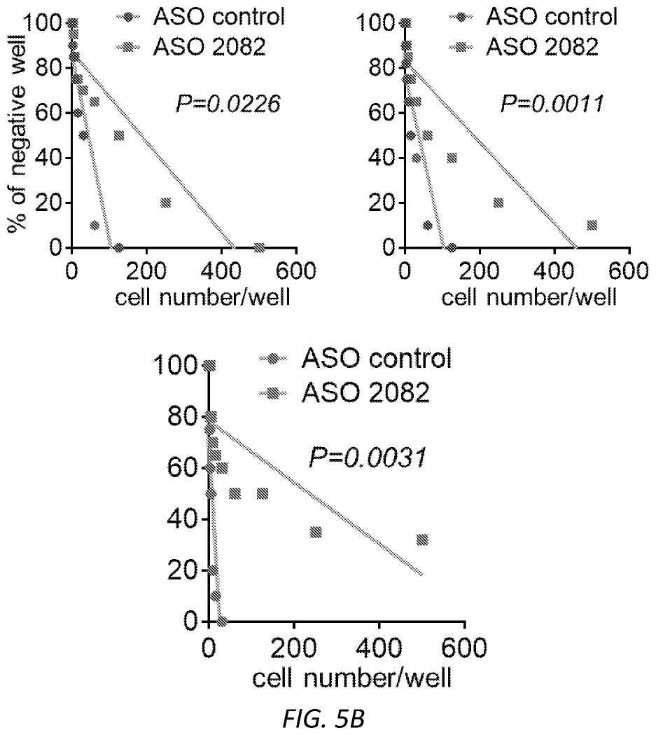

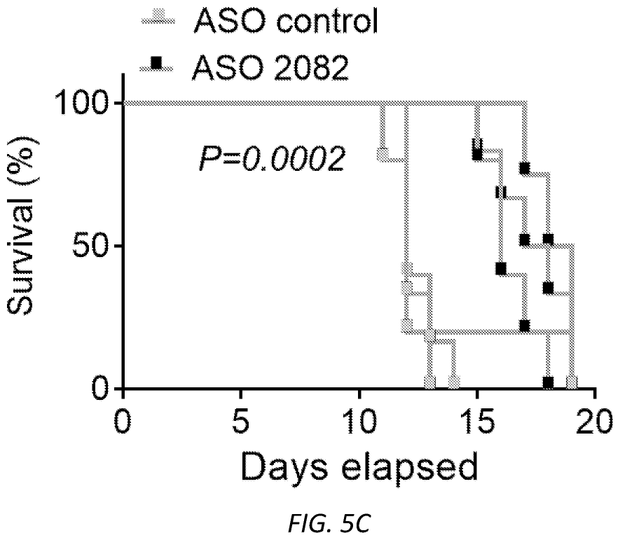

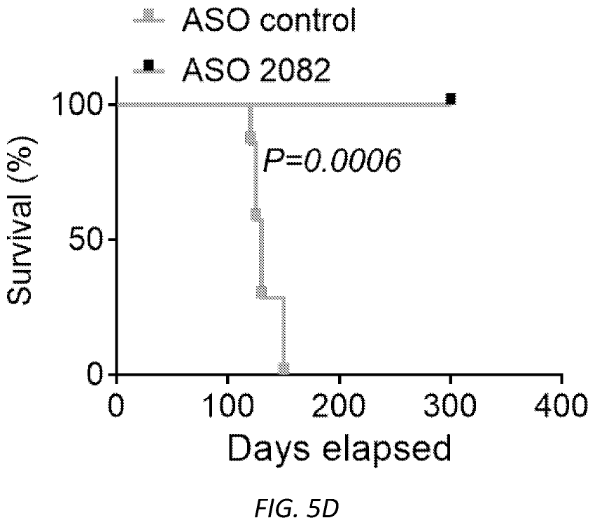

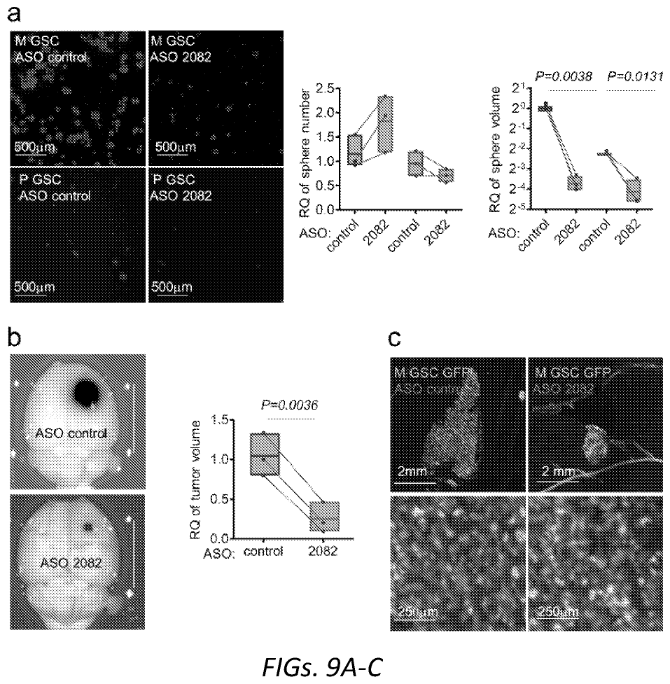

The efficient and specific knockdown of circ2082 allowed precise characterization of its phenotypic footprint in GSCs. In vitro, both limiting dilution assay and neurosphere formation assays demonstrated the strong anti-GSC effect of circ2082 knockdown (FIG. 5A-B, 9A), underlining pan-glioblastoma effects of circ2082, as its knock-down affected equally both tested GSC subtypes, regardless of their very different transcriptomes. For testing in vivo effects of circ2082, GSCs were pre-treated with ASOs and implanted into the brains of athymic mice. Similar to in vitro observations, ensuing tumors were significantly smaller when M GSCs were treated with ASO circ2082 compared to scrambled control ones (FIGS. 9B-C). The analysis of tumor volume in P GSC-originated tumors was not feasible as these do not form nodular tumors but are very diffused, as was demonstrated previously (11). We also observed significant survival benefits of circ2082 knock-down. Mice bearing very aggressive M GSC-originated tumors showed an increase in median survival from 12 to 17 days, i.e., more than 40% of the post-implantation time, upon circ2082 knock-down. Implantation of P GSC-originated tumors pre-treated with ASO circ2082 did not result in mortality up to 300 days, suggesting the inhibitory effect of the knock-down during the tumor initiation phase (FIGS. 5C-D). These results indicated that transcriptome deregulated by circ2082 in both subtypes might be critical for the observed phenotypes. Thus, to discover the culprits, we analyzed the fraction of the transcriptome deregulated in both subtypes. Even though 377 genes were significantly deregulated in both subtypes, they had no significant power to overcome the clustering of knock-down cells with their parental counterparts. Thus, to set up a circ2082-dependent signature into a broader clinical context, we queried these genes with the TCGA glioblastoma patient dataset. It allowed us to filter out six genes (DCX, STMN1, MAPT, LCP2, VAMP5, and ANXA4) annotated with hazard ratios from Cox analysis, which were sufficient to cluster patients' samples accordingly with either up- or down-regulation of these genes upon circ2082 knockdown. These clusters had significant power to predict the outcome using Kaplan-Meier estimator survival analysis. These results thus identified and described the action of the effectors of circ2082-dependent phenotype in GSCs, yet the role of microRNAome de-regulation in these processes remained unsubstantiated. So instead of further scrutiny of the transcriptome itself, we re-visited microRNAome analysis to verify whether direct targets of selected microRNAs provide sufficient evidence for the explanation of a circ2082-dependent effect.

### Example 7. Clinical Significance of Circ2082 Knock-Down

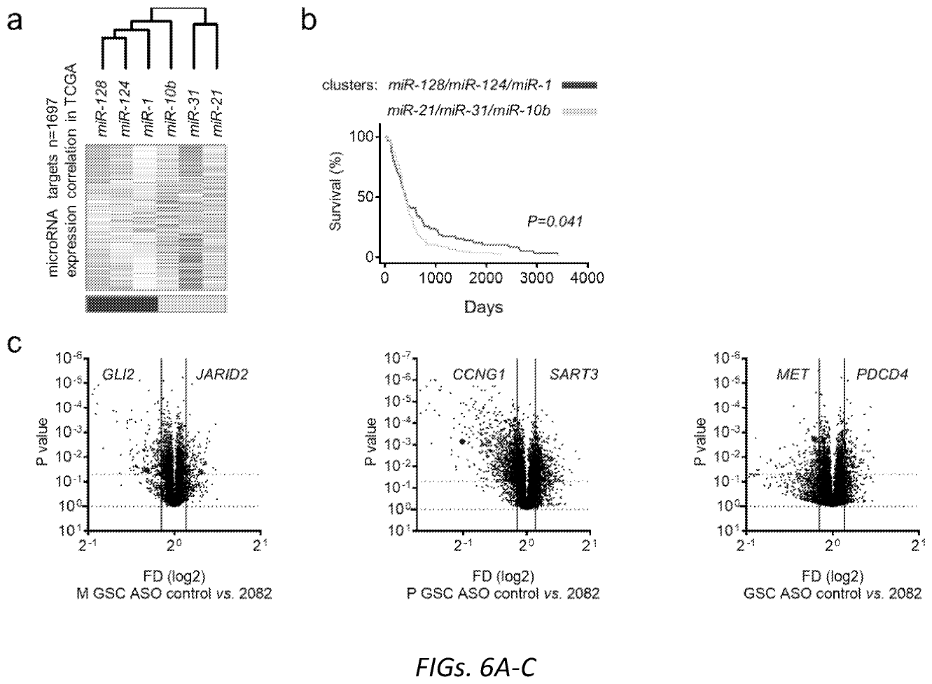

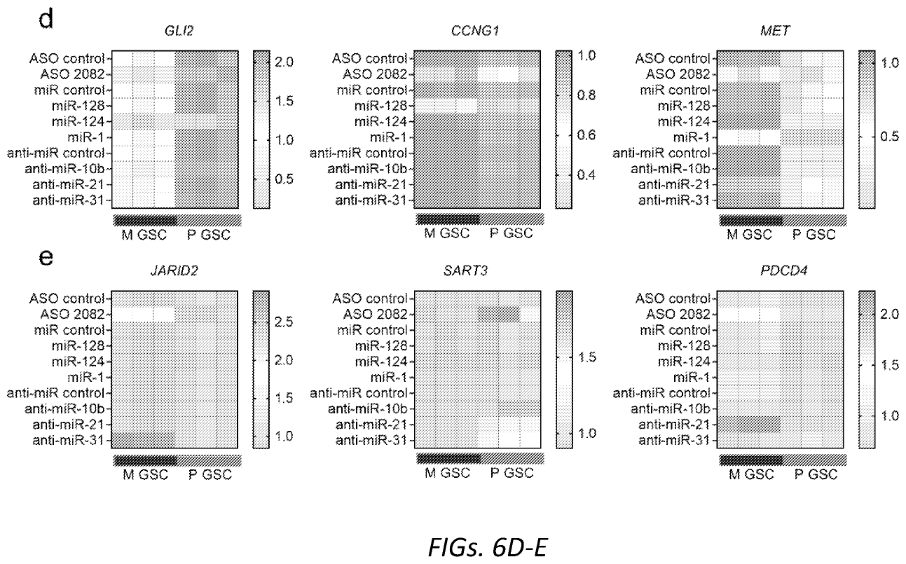

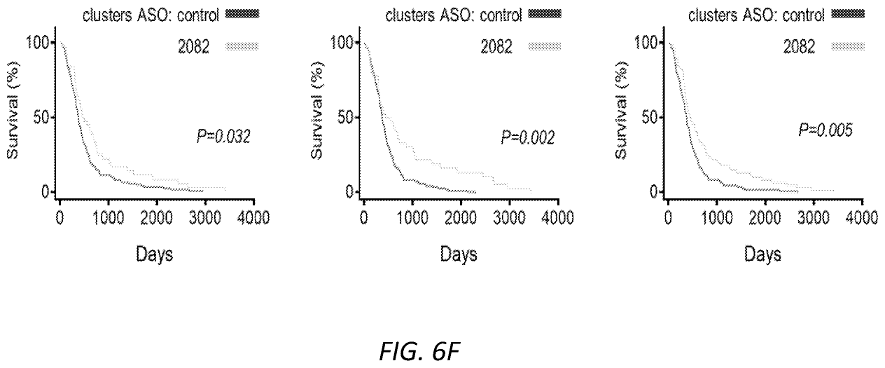

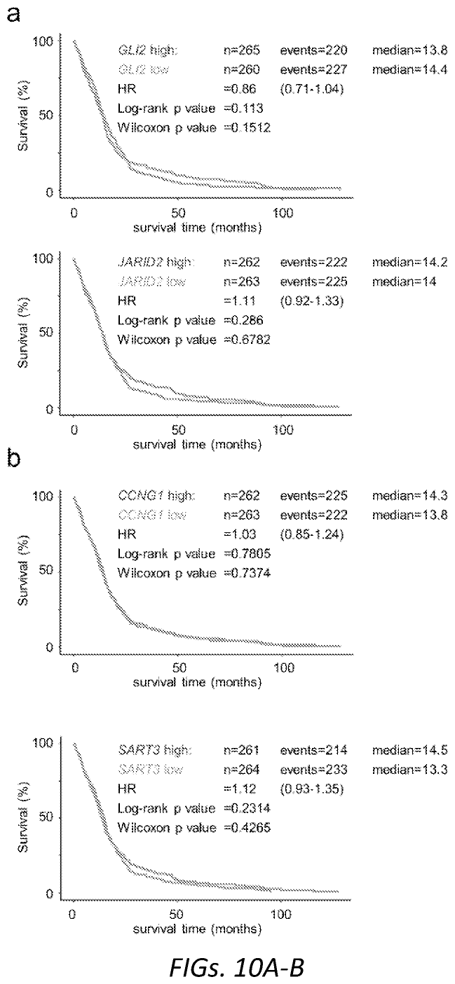

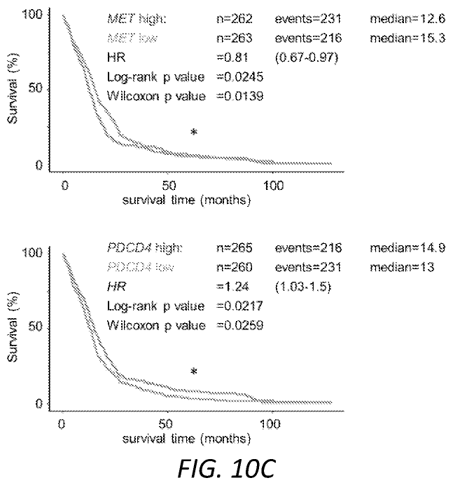

As a proof of concept, we first aimed to identify genes correlated with the expression of microRNAs selected previously as de-regulated in glioblastoma and being circ2082-dependent (see description of FIGS. 1A-B and 5A-D). To show a correlation of expression between genes and microRNAs, we re-visited the TCGA glioblastoma dataset. It revealed a correlation signature of ˜1700 genes that were either positively or negatively associated with microRNAs (FIG. 6A) and stratified the samples into two clusters: miR-128 miR-124, miR-1 (cluster 1), and miR-10b miR-31, miR-21 (cluster 2) with the power of outcome prediction (FIG. 6b). Yet, we had already demonstrated that the subtype identity remained the dominant force in this clustering (FIG. 5E-F). To circumvent this dominance in the analysis of microRNA-mediated circ2082-dependent effect, we thus supervised the analysis of previously identified microRNA clusters' mRNA targets. To this end, we separately scrutinized the expression of genes (from an initially selected set of ˜1700) after circ2082 knock-down in M GSCs only, in P GSCs only, and in both subtypes. Based on these results, we selected one previously validated, significantly altered mRNA target for each of 6 proof-of-concept microRNAs in all three scenarios to experimentally evaluate the effect of circ2082 knock-down-dependent de-repression of microRNAome (FIGS. 6C-E, respectively). These included: in M GSC circ2082 knock-down cells GLI2—a down-regulated target of miR-124 (29) and JARID2—an up-regulated target of miR-31 (30); in P GSC circ2082 knock-down cells CCNG1—a down-regulated target of miR-128 (31) and SART3—an up-regulated target of miR-10b (32); and in both GSCs circ2082 knock-down cells MET—a down-regulated target of miR-1 (21) and PDCD4—an up-regulated target of miR-21 (33). We compared their expression upon i) circ2082 knock-down, ii) exogenous overexpression of depleted microRNAs, and iii) antisense-mediated knock-down of highly expressed microRNAs. The apparent expression pattern revealed that genes repressed after circ2082 knock-down were also suppressed in response to exogenous overexpression of their respective microRNAs in both GSC subtypes (FIG. 6D). On the contrary, genes maintained after circ2082 knock-down were de-repressed in response to the inhibition of their “master” microRNAs only in the scenario when these microRNAs and mRNAs were expressed in control cells (FIG. 6E). These results suggested that the de-repression of microRNAome by circ2082 knock-down had a strong cell type-independent effect, while the circ2082 knock-down-associated repression of oncogenic microRNAs was strictly cell type-specific. Although the one-by-one analysis of microRNA/mRNA pairs presented above was useful as a proof of concept, it is unlikely that these few tandems are responsible for the observed far-reaching phenotype, as few of them are significantly outcome-predictive (FIGS. 10A-C). Thus finally, to delineate a circ2082-dependent microRNA-associated signature in the clinical context, we queried microRNA correlation genes with the TCGA glioblastoma patient dataset. Genes deregulated in P GSCs, M GSCs, and all GSCs together were filtered to keep the most varied molecules amongst the list annotated with hazard ratios from Cox analysis. All three analyses had significant power to predict the outcome using Kaplan-Meier estimator survival analysis. These results thus identified and clarified the circ2082-dependent phenotype in GSCs in the context of circ2082-implemented microRNAome surveillance and de-regulation.

### REFERENCES

1. J. Alles et al., An estimate of the total number of true human miRNAs. Nucleic acids research 47, 3353-3364 (2019).
2. A. Helwak, G. Kudla, T. Dudnakova, D. Tollervey, Mapping the human miRNA interactome by CLASH reveals frequent noncanonical binding. Cell 153, 654-665 (2013).
3. B. P. Lewis, I. H. Shih, M. W. Jones-Rhoades, D. P. Bartel, C. B. Burge, Prediction of mammalian microRNA targets. Cell 115, 787-798 (2003).
4. S. Cheloufi, C. O. Dos Santos, M. M. Chong, G. J. Hannon, A dicer-independent miRNA biogenesis pathway that requires Ago catalysis. Nature 465, 584-589 (2010).
5. K. N. Ivey, D. Srivastava, MicroRNAs as regulators of differentiation and cell fate decisions. Cell stem cell 7, 36-41 (2010).
6. J. Lu et al., MicroRNA expression profiles classify human cancers. Nature 435, 834-838 (2005).
7. D. V. Brown, S. S. Stylli, A. H. Kaye, T. Mantamadiotis, Multilayered Heterogeneity of Glioblastoma Stem Cells: Biological and Clinical Significance. Advances in experimental medicine and biology 1139, 1-21 (2019).
8. C. Neftel et al., An Integrative Model of Cellular States, Plasticity, and Genetics for Glioblastoma. Cell 178, 835-849 e821 (2019).
9. P. Mao et al., Mesenchymal glioma stem cells are maintained by activated glycolytic metabolism involving aldehyde dehydrogenase 1A3. Proc Natl Acad Sci USA 110, 8644-8649 (2013).
10. A. P. Patel et al., Single-cell RNA-seq highlights intratumoral heterogeneity in primary glioblastoma. Science 344, 1396-1401 (2014).
11. A. K. Rooj et al., MicroRNA-Mediated Dynamic Bidirectional Shift between the Subclasses of Glioblastoma Stem-like Cells. Cell Rep 19, 2026-2032 (2017).
12. J. Kim et al., Spatiotemporal Evolution of the Primary Glioblastoma Genome. Cancer cell 28, 318-328 (2015).
13. M. Mineo et al., The Long Non-coding RNA HIF1A-AS2 Facilitates the Maintenance of Mesenchymal Glioblastoma Stem-like Cells in Hypoxic Niches. Cell Rep 15, 2500-2509 (2016).
14. B. Kleaveland, C. Y. Shi, J. Stefano, D. P. Bartel, A Network of Noncoding Regulatory RNAs Acts in the Mammalian Brain. Cell 174, 350-362 e317 (2018).
15. S. Memczak et al., Circular RNAs are a large class of animal RNAs with regulatory potency. Nature 495, 333-338 (2013).
16. A. Rybak-Wolf et al., Circular RNAs in the Mammalian Brain Are Highly Abundant, Conserved, and Dynamically Expressed. Molecular cell 58, 870-885 (2015).
17. Y. Enuka et al., Circular RNAs are long-lived and display only minimal early alterations in response to a growth factor. Nucleic acids research 44, 1370-1383 (2016).
18. T. B. Hansen et al., Natural RNA circles function as efficient microRNA sponges. Nature 495, 384-388 (2013).
19. L. S. Kristensen, T. B. Hansen, M. T. Veno, J. Kjems, Circular RNAs in cancer: opportunities and challenges in the field. Oncogene 37, 555-565 (2018).
20. J. Silber et al., miR-124 and miR-137 inhibit proliferation of glioblastoma multiforme cells and induce differentiation of brain tumor stem cells. BMC medicine 6, 14 (2008).
21. A. Bronisz et al., Extracellular vesicles modulate the glioblastoma microenvironment via a tumor suppression signaling network directed by miR-1. Cancer Res 74, 738-750 (2014).
22. T. Papagiannakopoulos, A. Shapiro, K. S. Kosik, MicroRNA-21 targets a network of key tumor-suppressive pathways in glioblastoma cells. Cancer Res 68, 8164-8172 (2008).
23. V. Bhaskaran et al., The functional synergism of microRNA clustering provides therapeutically relevant epigenetic interference in glioblastoma. Nature communications 10, 442 (2019).
24. J. Godlewski et al., MicroRNA Signatures and Molecular Subtypes of Glioblastoma: The Role of Extracellular Transfer. Stem Cell Reports 8, 1497-1505 (2017).
25. A. Kurzynska-Kokorniak et al., The many faces of Dicer: the complexity of the mechanisms regulating Dicer gene expression and enzyme activities. Nucleic acids research 43, 4365-4380 (2015).
26. J. Pilotte, E. E. Dupont-Versteegden, P. W. Vanderklish, Widespread regulation of miRNA biogenesis at the Dicer step by the cold-inducible RNA-binding protein, RBM3. PloS one 6, e28446 (2011).
27. R. B. Zhou, X. L. Lu, C. Y. Zhang, D. C. Yin, RNA binding motif protein 3: a potential biomarker in cancer and therapeutic target in neuroprotection. Oncotarget 8, 22235-22250 (2017).
28. D. J. Voce et al., Temozolomide Treatment Induces lncRNA MALAT1 in an NF-kappaB and p53 Codependent Manner in Glioblastoma. Cancer Res 79, 2536-2548 (2019).
29. L. Xu et al., Inhibition of the Hedgehog signaling pathway suppresses cell proliferation by regulating the Gli2/miR-124/AURKA axis in human glioma cells. International journal of oncology 50, 1868-1878 (2017).
30. Y. Xue et al., Direct conversion of fibroblasts to neurons by reprogramming PTB-regulated microRNA circuits. Cell 152, 82-96 (2013).
31. M. Li et al., miR-128 and its target genes in tumorigenesis and metastasis. Exp Cell Res 319, 3059-3064 (2013).
32. N. M. Teplyuk et al., Therapeutic potential of targeting microRNA-10b in established intracranial glioblastoma: first steps toward the clinic. EMBO molecular medicine 8, 268-287 (2016).
33. M. Zhang et al., Nestin and CD133: valuable stem cell-specific markers for determining clinical outcome of glioma patients. J Exp Clin Cancer Res 27, 85 (2008).
34. H. de The, Differentiation therapy revisited. Nature reviews. Cancer 18, 117-127 (2018).
35. E. Anastasiadou, L. S. Jacob, F. J. Slack, Non-coding RNA networks in cancer. Nature reviews. Cancer 18, 5-18 (2018).
36. N. Passon et al., Expression of Dicer and Drosha in triple-negative breast cancer. J Clin Pathol 65, 320-326 (2012).
37. C. Much et al., Endogenous Mouse Dicer Is an Exclusively Cytoplasmic Protein. PLoS Genet 12, e1006095 (2016).
38. T. Treiber, N. Treiber, G. Meister, Regulation of microRNA biogenesis and its crosstalk with other cellular pathways. Nature reviews. Molecular cell biology 20, 5-20 (2019).
39. L. Verduci, S. Strano, Y. Yarden, G. Blandino, The circRNA-microRNA code: emerging implications for cancer diagnosis and treatment. Molecular oncology 13, 669-680 (2019).
40. M. Zhao et al., MALAT1: A long non-coding RNA highly associated with human cancers. Oncology letters 16, 19-26 (2018).
41. D. Ogawa et al., MicroRNA-451 Inhibits Migration of Glioblastoma while Making It More Susceptible to Conventional Therapy. Noncoding RNA 5, (2019).
42. K. I. Ansari et al., Glucose-based regulation of miR-451/AMPK signaling depends on the OCT1 transcription factor. Cell Rep 11, 902-909 (2015).
43. J. Godlewski et al., MicroRNA-451 regulates LKB1/AMPK signaling and allows adaptation to metabolic stress in glioma cells. Molecular cell 37, 620-632 (2010).
44. A. Zirkel, A. Papantonis, Detecting Circular RNAs by RNA Fluorescence In Situ Hybridization. Methods Mol Biol 1724, 69-75 (2018).
45. J. Godlewski et al., Targeting of the Bmi-1 oncogene/stem cell renewal factor by microRNA-128 inhibits glioma proliferation and self-renewal. Cancer Res 68, 9125-9130 (2008).
46. K. Meyer et al., Direct conversion of patient fibroblasts demonstrates non-cell autonomous toxicity of astrocytes to motor neurons in familial and sporadic ALS. Proc Natl Acad Sci USA 111, 829-832 (2014).
47. C. A. Schneider, W. S. Rasband, K. W. Eliceiri, NIH Image to ImageJ: 25 years of image analysis. Nat Methods 9, 671-675 (2012).
48. R. L. Bowman, Q. Wang, A. Carro, R. G. Verhaak, M. Squatrito, GlioVis data portal for visualization and analysis of brain tumor expression datasets. Neuro Oncol 19, 139-141 (2017).
49. O. Celiku, S. Johnson, S. Zhao, K. Camphausen, U. Shankavaram, Visualizing molecular profiles of glioblastoma with GBM-BioDP. PLoS One 9, e101239 (2014).
50. Y. Chen et al., Expression of amyloid precursor-like protein 2 (APLP2) in glioblastoma is associated with patient prognosis. Folia Neuropathol 56, 30-38 (2018).
51. L. M. Orre et al., SubCellBarCode: Proteome-wide Mapping of Protein Localization and Relocalization. Mol Cell 73, 166-182 e167 (2019).
52. R. G. Verhaak et al., Integrated genomic analysis identifies clinically relevant subtypes of glioblastoma characterized by abnormalities in PDGFRA, IDH1, EGFR, and NF1. Cancer Cell 17, 98-110 (2010).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An inhibitory nucleic acid, preferably of 10-50 nucleotides, comprising a sequence complementary to at least 10 consecutive nucleotides (nts) of SEQ ID NO:37, preferably comprising a sequence complementary to at least nts 48-49 or 47-50 of SEQ ID NO:37, plus additional nts on one or both ends.

2. The inhibitory nucleic acid of claim 1, wherein the inhibitory nucleic acid comprises at least 10, 11, 12, 13, 14, 15, 16, 17, 18, 19, or all 20 consecutive nucleotides of
(SEQ ID NO: 33)
GTTTCTAAAAATACACCAGC.

3. The inhibitory nucleic acid of claim 1, which is or comprises an antisense RNA oligonucleotide; antisense DNA oligonucleotide; chimeric antisense oligonucleotide; short, hairpin RNA (shRNA); or single- or double-stranded short interfering RNA (siRNA) for RNA interference (RNAi).

4. The inhibitory nucleic acid of claim 3, comprising one or more modifications.

5. The inhibitory nucleic acid of claim 4, wherein the one or more modifications comprise one or more modified bonds or bases, and/or conjugation to one or more moieties or conjugates that enhance the activity, cellular distribution, or cellular uptake of the oligonucleotide.

6. The inhibitory nucleic acid of claim 5, wherein the modified bonds comprise amide backbone; morpholino backbone; or peptide nucleic acid (PNA) backbone; wherein the modified bases comprise locked nucleic acids, phosphorothioate, methylphosphonate, peptide nucleic acids; and/or the conjugated moiety is a cholesterol, α-tocopherol, polyethulene glycol (PEG), biotin, or nanoparticle.

7. The inhibitory nucleic acid of claim 3, wherein the chimeric antisense oligonucleotide is a gapmer or mixmer or a DNA/RNA heteroduplex oligonucleotide (HDO).

8. The inhibitory nucleic acid of claim 1, comprising SEQ ID NO:33.

9. A pharmaceutical composition comprising the inhibitory nucleic acid of claim 1, and a pharmaceutically effective carrier.

10. A method of treating a cancer in a subject, the method comprising administering to the subject a therapeutically effective amount of an inhibitory nucleic acid, preferably of 10-50 nucleotides, comprising a sequence complementary to at least 10 consecutive nucleotides (nts) of SEQ ID NO:37, preferably comprising a sequence complementary to at least nts 48-49 or 47-50 of SEQ ID NO:37, plus additional nts on one or both ends.

11. The method of claim 10, wherein the inhibitory nucleic acid comprises at least 10, 11, 12, 13, 14, 15, 16, 17, 18, 19, or all 20 consecutive nucleotides of
(SEQ ID NO: 33)
GTTTCTAAAAATACACCAGC.

12. The method of claim 10, wherein the inhibitory nucleic acid is or comprises an antisense RNA oligonucleotide; antisense DNA oligonucleotide; chimeric antisense oligonucleotide; short, hairpin RNA (shRNA); or single- or double-stranded short interfering RNA (siRNA) for RNA interference (RNAi).

13. The method of claim 12, wherein the inhibitory nucleic acid comprises one or more modifications.

14. The method of claim 13, wherein the one or more modifications comprise one or more modified bonds or bases, and/or conjugation to one or more moieties or conjugates that enhance the activity, cellular distribution, or cellular uptake of the oligonucleotide.

15. The method of claim 5, wherein the modified bonds comprise amide backbone; morpholino backbone; or peptide nucleic acid (PNA) backbone; wherein the modified bases comprise locked nucleic acids, phosphorothioate, methylphosphonate, peptide nucleic acids; and/or the conjugated moiety is a cholesterol, α-tocopherol, polyethulene glycol (PEG), or biotin.

16. The method of claim 3, wherein the chimeric antisense oligonucleotide is a gapmer or mixmer or a DNA/RNA heteroduplex oligonucleotide (HDO).

17. The method of claim 10, wherein the inhibitory nucleic acid comprises SEQ ID NO:33.

18. The method of claim 10, wherein the cancer is a solid tumor.

19. The method of claim 18, wherein the cancer is brain cancer, optionally glioblastoma; breast; prostate; pancreatic; hepatic; lung; kidney; skin; head and neck; bladder; ovarian; or colon cancer.

20. (canceled)

21. (canceled)

22. (canceled)
